(19) United States
(12) Patent Application Publication
(10) Pub. No.: US 2020/0405809 A1
(43) Pub. Date: Dec. 31, 2020

(54) COMBINATION OF A CYCLIN DEPENDENT KINASE INHIBITOR AND A BET- BROMODOMAIN INHIBITOR

(71) Applicant: Pfizer Inc., New York, NY (US)

(72) Inventors: Lars Anders, San Diego, CA (US); Danan Li, San Diego, CA (US)

(73) Assignee: Pfizer Inc., New York, NY (US)

(21) Appl. No.: 16/975,837

(22) PCT Filed: Feb. 22, 2019
(86) PCT No.: PCT/IB2019/051462

§ 371 Date: Aug. 26, 2020

Related U.S. Application Data
(60) Provisional application No. 62/796,223, filed on Jan. 24, 2019.
(60) Provisional application No. 62/635,916, filed on Feb. 27, 2018.

(51) Int. Cl. A61K 38/17; A61K 31/519; A61K 31/437; A61K 31/5025; A61K 31/4196; A61K 31/565; A61P 35/00

(52) U.S. Cl. CPC A61K 38/1709; A61K 31/519; A61K 31/437; A61P 35/00; A61K 31/4196; A61K 31/565; A61K 31/5025

(57) ABSTRACT

This invention relates to combination therapies comprising a cyclin dependent kinase (CDK) inhibitor that inhibits CDK4 and/or CDK6, and a bromodomain and extra-terminal domain (BET) family inhibitor, and associated pharmaceutical compositions, methods of treatment, and pharmaceutical uses.

### BACKGROUND OF THE INVENTION

### Field of the Invention

The present invention relates to combination therapies useful for the treatment of cancer. In particular, the invention relates to combination therapies comprising an inhibitor of cyclin dependent kinase 4 (CDK4), cyclin dependent kinase 6 (CDK6) or cyclin dependent kinase 4 and 6 (CDK4/6), and an inhibitor of the bromodomain and extra-terminal domain (BET) family (BET inhibitor). The invention also relates to associated methods of treatment, pharmaceutical compositions, and pharmaceutical uses.

### Description of the Related Art

Epigenetics holds great promise for combination treatments in oncology (Jones et al., Targeting the cancer epigenome for therapy, Nat. Rev. Genetics (2016) 17: 630-641). Bromodomain-containing proteins are of significant biological interest, as components of transcription factor complexes and determinants of epigenetic memory. The BET family (BRD2, BRD3, BRD4 and BRDT) shares a common domain architecture featuring two amino-terminal bromodomains that exhibit high levels of sequence conservation, and a more divergent carboxy-terminal recruitment domain (Filippakopouloset al., Selective inhibition of BET bromodomains, Nature (2010) 468, 1067-1073). BRD2 and BRD3 are reported to associate with histones along actively transcribed genes and may be involved in facilitating transcriptional elongation (Leroy et al, The double bromodomain proteins Brd2 and Brd3 couple histone acetylation to transcription, Mol. Cell. (2008) 30, 51-60). It has also been reported that BRD4 or BRD3 may fuse with NUT (nuclear protein in testis) forming novel fusion oncogenes, BRD4-NUT or BRD3-NUT, in a highly malignant form of epithelial neoplasia (French et al. BRD4-NUT fusion oncogene: a novel mechanism in aggressive carcinoma, Cancer Res., (2003) 63:304-307 and French et al. Midline carcinoma of children and young adults with NUT rearrangement, J. Clin. Oncol. (2004) 22, 4135-4139). Data suggests that BRD-NUT fusion proteins contribute to carcinogenesis (French et al. BRD-NUT oncoproteins: a family of closely related nuclear proteins that block epithelial differentiation and maintain the growth of carcinoma cells, Oncogene (2008) 27, 2237-2242). To date, BRDT is thought to be uniquely expressed in the testes and ovary. All family members have been reported to have some function in controlling or executing aspects of the cell cycle, and have been shown to remain in complex with chromosomes during cell division, suggesting a role in the maintenance of epigenetic memory. In addition, some viruses make use of these proteins to tether their genomes to the host cell chromatin, as part of the process of viral replication (You et al. Interaction of the bovine papillomavirus E2 protein with Brd4 tethers the viral DNA to host mitotic chromosomes, Cell (2004) 117, 349-60). BRD4 appears to be involved in the recruitment of the pTEF-P complex to inducible genes, resulting in phosphorylation of RNA polymerase and increased transcriptional output (Hargreaves et al, Control of inducible gene expression by signal-dependent transcriptional elongation, Cell (2009) 138:129-145). BRD-4 has also been shown to bind to acetylated lysine-310 of the RelA subunit of NF-κB resulting in enhanced transcriptional activation of NF-κB and the expression of a subset of NF-κB responsive inflammatory genes (Huang et al, Brd4 Coactivates Transcriptional Activation of NF-κB via Specific Binding to Acetylated RelA, Mol Cell Biol (2009) 29 1375-1387).

Bromodomain-containing protein 4 (BRD4) is a member of the BET family that, in yeast and animals, contains two tandem bromodomains (BD1 and BD2) and an extraterminal (ET) domain. BRD4 is a double bromodomain-containing protein that binds preferentially to acetylated chromatin and acetylated lysine-310 of the RelA subunit of NF-κB. In humans, four BET proteins (BRD2, BRD3, BRD4 and BRDT) exhibit similar gene arrangements, domain organizations, and some functional properties (Wu and Chiang, The Double Bromodomain-containing Chromatin Adaptor Brd4 and Transcriptional Regulation, J. Biol. Chem. (2007) 282:13141-13145).

Cyclin-dependent kinases (CDKs) and related serine/threonine protein kinases are important cellular enzymes that perform essential functions in regulating cell division and proliferation. CDKs 1-4, 6, 10, 11 have been reported to play a direct role in cell cycle progression, while CDKs 3, 5 and 7-9 may play an indirect role (e.g., through activation of other CDKs, regulation of transcription or neuronal functions). The CDK catalytic units are activated by binding to regulatory subunits, known as cyclins, followed by phosphorylation. Cyclins can be divided into four general classes (G₁, G₁/S, S and M cyclins) whose expression levels vary at different points in the cell cycle. Cyclin B/CDK1, cyclin A/CDK2, cyclin E/CDK2, cyclin D/CDK4, cyclin D/CDK6, and likely other heterodynes are important regulators of cell cycle progression.

CDK inhibitors have been demonstrated to be useful in treating cancer. Increased activity or temporally abnormal activation of cyclin-dependent kinases has been shown to result in the development of human tumors, and human tumor development is commonly associated with alterations in either the CDK proteins themselves or their regulators (Cordon-Cardo C. Mutations of cell cycle regulators: biological and clinical implications for human neoplasia. Am. J. Pathol. (1995) 147:545-560; Karp J E, Broder S. Molecular foundations of cancer: new targets for intervention. Nat. Med. (1995) 1:309-320; Hall M, Peters G. Genetic alterations of cyclins, cyclin-dependent kinases, and Cdk inhibitors in human cancer. Adv. Cancer Res. (1996) 68:67-108).

Mutations of CDK4 and CDK6 have been described in subgroups of melanoma and other tumors (Zuo L, et al., Germ line mutations in the p16INK4a binding domain of CDK4 in familial melanoma. Nature Genet. (1996) 12, 97-99; Ortega S, et al. Cyclin D-dependent kinases, INK4 inhibitors and cancer. Biochim. Biophys. Acta (2002) 1602:73-87; Smalley KSM et al. Identification of a novel subgroup of melanomas with KIT/cyclin-dependent kinase-4 overexpression. Cancer Res (2008) 68: 5743-52). Amplifications of the regulatory subunits of CDKs and cyclins, and mutation, gene deletion, or transcriptional silencing of endogenous INK4 CDK inhibitors have also been reported as mechanism by which the pathway can be activated (Smalley KSM (2008)). The use of CDK4/6 inhibitors in combination with endocrine therapy has demonstrated significant efficacy in the treatment of hormone receptor (HR)-positive, human epidermal growth factor 2 (HER2)-negative advanced or metastatic breast cancers, and CDK4/6 inhibitors have been approved in combination with aromatase inhibitors in a first-line setting and fulvestrant in a second-line setting.

Nevertheless, there remains a need for improved therapies for the treatment of cancers. The combinations and methods of the present invention are believed to have one or more advantages, such as greater efficacy than treatment with either therapeutic agent alone; potential to reduce drug-drug interactions; potential to enable an improved dosing schedule; potential to reduce side effects; potential to overcome resistance mechanisms and the like. These, and other advantages of the present invention, are apparent from the description below.

### BRIEF SUMMARY OF THE INVENTION

This invention relates to therapeutic methods, combinations and compositions for use in the treatment of abnormal cell growth, particularly cancer.

In one aspect, the invention provides a method of treating cancer in a subject comprising administering to the subject an effective amount of a cyclin dependent kinase (CDK) inhibitor and an effective amount of a bromodomain and extra-terminal domain (BET) inhibitor, wherein the CDK inhibitor is an inhibitor of cyclin dependent kinase 4 (CDK4), cyclin dependent kinase 6 (CDK6), or both CDK4 and CDK6 (CDK4/6).

In another aspect, the invention provides a combination comprising a CDK inhibitor and a BET inhibitor, wherein the CDK inhibitor is a CDK4 inhibitor, a CDK6 inhibitor, or a CDK4/6 inhibitor. In some embodiments, the combination is useful for the treatment of cancer in a subject. In some embodiments, the combination is a synergistic combination.

In another aspect, the invention provides use of a combination comprising a CDK inhibitor and a BET inhibitor for the treatment of cancer in a subject, wherein the CDK inhibitor is a CDK4 inhibitor, a CDK6 inhibitor or a CDK4/6 inhibitor. In some embodiments, the combination is a synergistic combination.

In a further aspect, the invention provides a pharmaceutical composition comprising a CDK inhibitor, a BET inhibitor, and a pharmaceutically acceptable carrier or excipient, wherein the CDK inhibitor is a CDK4 inhibitor, a CDK6 inhibitor, or a CDK4/6 inhibitor.

In certain embodiments, the CDK inhibitor is a CDK4/6 inhibitor. In some such embodiments, the CDK4/6 inhibitor is selected from the group consisting of palbociclib, ribociclib and abemaciclib, or a pharmaceutically acceptable salt thereof. In particular embodiments, the CDK4/6 inhibitor is palbociclib, or a pharmaceutically acceptable salt thereof.

In some embodiments, the BET inhibitor is an inhibitor of one or more of bromodomain-containing protein 2 (BRD2), bromodomain-containing protein 3 (BRD3), bromodomain-containing protein 4 (BRD4), or testis-specific bromodomain-containing protein (BRDT).

In certain embodiments, the BET inhibitor is a BRD4 inhibitor, a BRD2 inhibitor, and/or a BRD2/4 inhibitor. In some embodiments, the BET inhibitor is a BRD4 inhibitor.

In some such embodiments, the BET inhibitor further inhibits BRD2 and/or BRDT. In particular embodiments, the BET inhibitor is mivebresib or AZD5153, or a pharmaceutically acceptable salt thereof.

In a preferred embodiment, the CDK4/6 inhibitor is palbociclib, or a pharmaceutically acceptable salt thereof and the BET inhibitor is mivebresib, or a pharmaceutically acceptable salt thereof. In another preferred embodiment, the CDK4/6 inhibitor is palbociclib, or a pharmaceutically acceptable salt thereof and the BET inhibitor is AZD5153, or a pharmaceutically acceptable salt thereof.

Each of the aspects and embodiments of the present invention described below may be combined with one or more other embodiments of the present invention described herein which is not inconsistent with the embodiment(s) with which it is combined. In addition, each of the embodiments below describing the invention envisions within its scope the pharmaceutically acceptable salts of the compounds of the invention. Accordingly, the phrase “or a pharmaceutically acceptable salt thereof” is implicit in the description of all compounds described herein.

### BRIEF DESCRIPTION OF THE DRAWINGS

FIG. 1 shows a schematic diagram for screening test combinations in T47D breast cancer tumor spheroids using area-under-curve analysis to determine synergy.

FIGS. 2A, 2B and 2C show enhanced growth inhibition by palbociclib and BET inhibitor B1 combination in T47D breast cancer spheroids, as average diameter (mm) at concentrations shown.

FIGS. 3A, 3B and 3C show enhanced growth inhibition by palbociclib and BET inhibitor B2 combination in T47D breast cancer spheroids, as average diameter (mm) at concentrations shown.

FIGS. 4A, 4B and 4C show enhanced growth inhibition by palbociclib and BET inhibitor B3 combination in T47D breast cancer spheroids, as average diameter (mm) at concentrations shown.

FIGS. 5A, 5B and 5C show enhanced growth inhibition by palbociclib and BET inhibitor B4 combination in T47D breast cancer spheroids, as average diameter (mm) at concentrations shown.

FIGS. 6A, 6B and 6C show enhanced growth inhibition by palbociclib and BET inhibitor B5 combination in T47D breast cancer spheroids, as average diameter (mm) at concentrations shown.

FIGS. 7A, 7B and 7C show enhanced growth inhibition by palbociclib and BET inhibitor B6 combination in T47D breast cancer spheroids, as average diameter (mm) at concentrations shown.

FIGS. 8A, 8B and 8C show enhanced growth inhibition by palbociclib and BET inhibitor B7 combination in T47D breast cancer spheroids, as average diameter (mm) at concentrations shown.

FIGS. 9A, 9B and 9C show enhanced growth inhibition by palbociclib and BET inhibitor B8 combination in T47D breast cancer spheroids, as average diameter (mm) at concentrations shown.

FIGS. 10A, 10B and 10C show enhanced growth inhibition by palbociclib and BET inhibitor B9 combination in T47D breast cancer spheroids, as average diameter (mm) at concentrations shown.

FIGS. 11A, 11B and 11C show enhanced growth inhibition by palbociclib and BET inhibitor B10 combination in T47D breast cancer spheroids, as average diameter (mm) at concentrations shown.

FIGS. 12A, 12B and 12C show enhanced growth inhibition by palbociclib and BET inhibitor B7 combination in Hs766T pancreatic cancer spheroids, as average diameter (mm) at concentrations shown.

FIGS. 13A, 13B and 13C show enhanced growth inhibition by palbociclib and BET inhibitor B8 combination in Hs766T pancreatic cancer spheroids, as average diameter (mm) at concentrations shown.

FIG. 14 shows that combination of palbociclib (50 mpk) and ABBV-075 (B7) (2 mpk) enhanced tumor growth delay and tumor growth inhibition in the MCF-7 breast cancer xenograft model. Dosing was stopped 21 days post-treatment initiation and tumors were allowed to recover until day 47; mean tumor volumes (mm³) are averages of MCF-7 xenografts (n=10).

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

The term “about” which used to modify a numerically defined parameter means that the parameter may vary by as much as 10% above or below the stated numerical value for that parameter. For example a dose of about 5mg/kg should be understood to mean that the dose may vary between 4.5 mg/kg and 5.5 mg.kg.

The term “administration” and “treatment” as it applies to an animal, human, experimental subject, cell, tissue, organ or biological fluid, refers to contact of an exogenous pharmaceutical, therapeutic or diagnostic agent, or composition, to the animal, human, experimental subject, cell, tissue, organ or biological fluid. Treatment of a cell encompasses contact of a reagent to the cell, as well as contact of a reagent to a fluid, where the fluid is in contact with the cell. “Administration” and “treatment” also means in vitro and ex vivo treatment, e.g., of a cell, by a reagent, diagnostic, binding compound, or by another cell.

The terms “abnormal cell growth” and “hyperproliferative disorder” are used interchangeably in this application.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous).

The term “cancer”, “cancerous”, “malignant” refer to or describe the physiological condition in mammals that is typically characterized by unregulated cell growth. As used herein “cancer” refers to any malignant and/or invasive growth or tumor caused by abnormal cell growth. As used herein “cancer” refers to solid tumors named for the type of cells that form them, cancer of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from latter one.

The term “patient” or “subject” refer to any single subject for which therapy is desired or that is participating in a clinical trial, epidemiological study or used as a control, including humans and mammalian veterinary patients such as cattle, horses, dogs and cats. In certain preferred embodiments, the subject is a human.

The term “treat” or “treating” a cancer as used herein means to administer a combination therapy according to the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of a tumor; remission of the cancer; decreasing symptoms resulting from the cancer; increasing the quality of life of those suffering from the cancer; decreasing the dose of other medications required to treat the cancer; delaying the progression of the cancer; curing the cancer; overcoming one or more resistance mechanisms of the cancer; and/or prolonging survival of patients the cancer. Positive therapeutic effects in cancer can be measured in a number of ways (see, for example, W. A. Weber, Assessing tumor response to therapy, J. Nucl. Med. 50 Suppl. 1:1S-10S (2009). For example, with respect to tumor growth inhibition (T/C), according to the National Cancer Institute (NCI) standards, a T/C less than or equal to 42% is the minimum level of anti-tumor activity. A T/C<10% is considered a high anti-tumor activity level, with T/C (%)=median tumor volume of the treated/median tumor volume of the control×100.

In some embodiments, the treatment achieved by a combination of the invention is defined by reference to any of the following: partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow, and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naïve or untreated subjects or patients. In some embodiments, response to a combination of the invention is any of PR, CR, PFS, DFS, OR or OS that is assessed using Response Evaluation Criteria in Solid Tumors (RECIST) 1.1 response criteria.

The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi2-test the U-test according to Mann and Whitney, the Kruskal-Wallis test (H-test), Jonckheere-Terpstrat-test and the Wilcon on-test.

The terms “treatment regimen”, “dosing protocol” and “dosing regimen” are used interchangeably to refer to the dose and timing of administration of each therapeutic agent in a combination of the invention.

“Ameliorating” means a lessening or improvement of one or more symptoms upon treatment with a combination described herein, as compared to not administering the combination. “Ameliorating” also includes shortening or reduction in duration of a symptom.

As used herein, an “effective dosage” or “effective amount” of drug, compound or pharmaceutical composition is an amount sufficient to effect any one or more beneficial or desired, including biochemical, histological and/or behavioral symptoms, of the disease, its complications and intermediate pathological phenotypes presenting during development of the disease. For therapeutic use, a “therapeutically effective amount” refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, (4) relieving to some extent (or, preferably, eliminating) one or more signs or symptoms associated with the cancer, (5) decreasing the dose of other medications required to treat the disease, and/or (6) enhancing the effect of another medication, and/or (7) delaying the progression of the disease in a patient.

An effective dosage can be administered in one or more administrations. For the purposes of this invention, an effective dosage of drug, compound, or pharmaceutical composition is an amount sufficient to accomplish prophylactic or therapeutic treatment either directly or indirectly. As is understood in the clinical context, an effective dosage of drug, compound or pharmaceutical composition may or may not be achieved in conjunction with another drug, compound or pharmaceutical composition.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas.

Examples of solid tumors are sarcomas, carcinomas, and lymphomas. Leukaemia's (cancers of the blood) generally do not form solid tumors (National Cancer Institute, Dictionary of Cancer Terms).

“Tumor burden” or “tumor load”, refers to the total amount of tumorous material distributed throughout the body. Tumor burden refers to the total number of cancer cells or the total size of tumor(s), throughout the body, including lymph nodes and bone marrow. Tumor burden can be determined by a variety of methods known in the art, such as, e.g., using callipers, or while in the body using imaging techniques, e.g., ultrasound, bone scan, computed tomography (CT), or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using callipers, or while in the body using imaging techniques, e.g., bone scan, ultrasound, CR or MRI scans.

The term “additive” is used to mean that the result of the combination of two compounds, components or targeted agents is no greater than the sum of each compound, component or targeted agent individually.

The term “synergy” or “synergistic” are used to mean that the result of the combination of two compounds, components or targeted agents is greater than the sum of each compound, component or targeted agent individually. This improvement in the disease, condition or disorder being treated is a “synergistic” effect. A “synergistic amount” is an amount of the combination of the two compounds, components or targeted agents that results in a synergistic effect, as “synergistic” is defined herein.

Determining a synergistic interaction between one or two components, the optimum range for the effect and absolute dose ranges of each component for the effect may be definitively measured by administration of the components over different dose ranges, and/or dose ratios to patients in need of treatment. However, the observation of synergy in in vitro models or in vivo models can be predictive of the effect in humans and other species and in vitro models or in vivo models exist, as described herein, to measure a synergistic effect. The results of such studies can also be used to predict effective dose and plasma concentration ratio ranges and the absolute doses and plasma concentrations required in humans and other species such as by the application of pharmacokinetic and/or pharmacodynamics methods.

CDK inhibitors useful in the invention include CDK4 inhibitors, CDK6 inhibitors, and CDK4/6 inhibitors. Such compounds may be pan-CDK inhibitors, which inhibit multiple CDKs, or may selectively inhibit CDK4 and/or CDK6. CDK inhibitors may have activity against targets in addition to CDKs. Such compounds may be identified using standard assays routinely used to measure inhibition of CDKs and other protein kinases. See, e.g., Fry et al., Cell cycle and biochemical effects of PD 0183812. A potent inhibitor of the cyclin D-dependent kinases CDK4 and CDK6, J. Biol. Chem. (2001), 276: 16617-16623. Typical CDK inhibitors have IC₅₀ values of less than 1 μM, preferably less than 100 nM, and more preferably less than 20 nM in such assays. The development of CDK inhibitors has been reviewed in the literature. For example, see Sanchez-Martinez et al. Cyclin dependent kinase (CDK) inhibitors as anticancer drugs, Bioorg. Med. Chem. Lett. (2015) 25: 3420-3435 (and references cited therein).

A number of CDK4/6 inhibitors have been approved or are currently in clinical development, including: palbociclib (also known as PD-0332991), ribociclib (also known as LEE-011), abemaciclib (also known as LY2835219), G1T38, trilaciclib (also known as GTI128) and SHR6390. Pan-CDK inhibitors having CDK4 activity include, but are not limited to AT7519, JNJ-7706621, P276-00, R547 (also known as RO-4584820), roniciclib (also known as BAY1000394), RGB-286638 and flavopiridol (alvocidib). Such compounds, or their pharmaceutically acceptable salts, may be useful in the present invention.

In some embodiments, the CDK inhibitor is a CDK4/6 inhibitor selected from the group consisting of palbociclib, ribociclib, abemaciclib, G1T38, trilaciclib and SHR6390, or a pharmaceutically acceptable salt thereof. In other embodiments, the CDK inhibitor is a CDK4/6 inhibitor selected from the group consisting of palbociclib, ribociclib and abemaciclib, or a pharmaceutically acceptable salt thereof. In specific embodiments, the CDK4/6 inhibitor is palbociclib, or a pharmaceutically acceptable salt thereof. In other embodiments, the CDK4/6 inhibitor is ribociclib, or a pharmaceutically acceptable salt thereof. In other embodiments, the CDK4/6 inhibitor is abemaciclib, or a pharmaceutically acceptable salt thereof. In further embodiments, the CDK4/6 inhibitor is G1T38, or a pharmaceutically acceptable salt thereof.

Palbociclib is described in WHO Drug Information, Vol. 27, No. 2, page 172 (2013). Palbociclib and pharmaceutically acceptable salts and formulations thereof are disclosed in International Publication No. WO 2003/062236 and U.S. Pat. Nos. 6,936,612, 7,208,489 and 7,456,168; International Publication No. WO 2005/005426 and U.S. Pat. Nos. 7,345,171 and 7,863,278; International Publication No. WO 2008/032157 and U.S. Pat. No. 7,781,583; International Publication No. WO 2014/128588; and International Publication No. WO 2016/193860. The contents of each of the foregoing references are incorporated herein by reference in their entirety.

BET inhibitors useful in the invention include, but are not limited to BRD4 inhibitors, BRD2 inhibitors and BRD2/4 inhibitors. Such compounds may be pan-BET inhibitors, which inhibit multiple BET targets, or may selectively inhibit BRD4 and/or BRD2. BET inhibitors may have activity against targets in addition to BETs. Such compounds may be identified using standard assays routinely used to measure binding to BET proteins. See, e.g., Zolotarjova, N. I. and. Wynn, R. Binding Assays for Bromodomain Proteins: Their Utility in Drug Discovery in Oncology and Inflammatory Disease, Current Protocols in Pharmacol. (2018), 80: 3.16.1-3.16.14. Typical BET inhibitors exhibit dose dependent inhibition of binding to the BET protein with IC₅₀ values of less than 1 μM, preferably less than 200 nM, and more preferably less than 50 nM.

The development of bromodomain BET inhibitors has been reviewed in the literature. For example, see Theodoulou et al. Clinical progress and pharmacology of small molecule bromodomain inhibitors, Curr. Opin. Chem. Biol. (2016), 33:58-66 (and references cited therein).

A number of BET inhibitors are currently in clinical development, including: ABBV-075 (also known as mivebresib); AZD5153; BAY1238097; BI-894999; BMS-986158; CPI-0610; FT-1101; GSK525762 (also known as IBET-762); GSK2820151; GS-5829; INCB054329; INCB057643; N-methyl-2-pyrrolidone; MK-8628 (OTX015); RO6870810 (also known as TEN-010); RVX-208 (also known as RVX000222 or apabetalone); and ZEN003694. Additional examples of BET inhibitors include, but are not limited to: IBET-151 (GSK1210151); JQ1; PFI-1; PFI-2; CPI-267203; IBET-819 (GW-841819X); BET-BAY-002; and SF-2523. Such compounds, or their pharmaceutically acceptable salts, may be useful in the present invention.

In some embodiments of the present invention, the BET inhibitor is selected from the group consisting of: mivebresib; AZD5153; BAY1238097; BI-894999; BMS-986158; CPI-0610; FT-1101; GSK525762; GSK2820151; GS-5829; INCB054329; INCB057643; N-methyl-2-pyrrolidone; MK-8628; RO6870810; apabetalone; and ZEN003694; or a pharmaceutically acceptable salt thereof. In some such embodiments, the BET inhibitor is mivebresib or AZD5153, or a pharmaceutically acceptable salt thereof. In other such embodiments, the BET inhibitor is mivebresib, or a pharmaceutically acceptable salt thereof. In other such embodiments, the BET inhibitor is AZD5153, or a pharmaceutically acceptable salt thereof.

Unless indicated otherwise, all references herein to CDK inhibitors and BET inhibitors include references to salts, solvates, hydrates and complexes thereof, and to solvates, hydrates and complexes of salts thereof, and include amorphous and polymorphic forms, stereoisomers, and isotopically labeled versions thereof. CDK inhibitors and BET inhibitors useful in the present invention may exist in the form of pharmaceutically acceptable salts such as, e.g., acid addition salts and base addition salts.

As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the compounds of the formulae disclosed herein. For example, the compounds of the invention that are basic in nature may be capable of forming a wide variety of salts with various inorganic and organic acids. The acids that may be used to prepare pharmaceutically acceptable acid addition salts of such basic compounds of those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, y-hydroxybutyrate, iodide, isobutyrate, isothionate, lactate, lactobionate, laurate, malate, maleate, malonate, mandelate, mesylate, metaphosphate, methylsulfate, monohydrogenphosphate, mucate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phospate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts. Alternatively the compounds useful that are acidic in nature may be capable of forming base salts with various pharmacologically acceptable cations. Examples of such salts include the alkali metal or alkaline-earth metal salts and particularly, the sodium and potassium salts. These salts may be prepared by conventional techniques. The chemical bases which may be used as reagents to prepare the pharmaceutically acceptable base salts of this invention include those which form non-toxic base salts with the acidic compounds herein. The chemical bases that may be used as reagents to prepare pharmaceutically acceptable base salts of the compounds of the invention that are acidic in nature are those that form non-toxic base salts with such compounds. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. Hemisalts of acids and bases may also be formed, for example, hem isulphate and hemicalcium salts. For a review on suitable salts, see Handbook of Pharmaceutical Salts: Properties, Selection, and Use by Stahl and Wermuth (Wiley-VCH, 2002). Methods for making pharmaceutically acceptable salts are known to those of skill in the art.

Further, the CDK inhibitors and BET inhibitors useful for the present invention may exist in both unsolvated and solvated forms. When the solvent or water is tightly bound, the complex will have a well-defined stoichiometry independent of humidity.

When, however, the solvent or water is weakly bound, as in channel solvates and hygroscopic compounds, the water/solvent content will be dependent on humidity and drying conditions. In such cases, non-stoichiometry will be the norm. The term ‘solvate’ is used herein to describe a molecular complex comprising the compound of the invention and one or more pharmaceutically acceptable solvent molecules, for example, ethanol. The term ‘hydrate’ is employed when the solvent is water. Pharmaceutically acceptable solvates in accordance with the invention include hydrates and solvates wherein the solvent of crystallization may be isotopically substituted, e.g. D₂O, d₆-acetone and d₆-DMSO.

The CDK inhibitors and BET inhibitors useful for the present invention may be used as crystalline or amorphous products, or mixtures thereof. They may be obtained, for example, as solid plugs, powders, or films by methods such as precipitation, crystallization, freeze drying, spray drying, or evaporative drying. Microwave or radio frequency drying may be used for this purpose.

### Therapeutic Methods, Uses, Combinations and Compositions

The methods, uses, combinations and compositions of the present inventions may be useful for treating cancer. Some embodiments provided herein result in one or more of the following effects: (1) inhibiting cancer cell proliferation; (2) inhibiting cancer cell invasiveness; (3) inducing apoptosis of cancer cells; (4) inhibiting cancer cell metastasis; (5) inhibiting angiogenesis; or (6) overcoming one or more resistance mechanisms relating to a cancer treatment.

In one aspect, the invention provides a method of treating cancer in a subject comprising administering to the subject a cyclin dependent kinase (CDK) inhibitor and a bromodomain and extra-terminal domain (BET) inhibitor, wherein the CDK inhibitor is an inhibitor of cyclin dependent kinase 4 (CDK4), cyclin dependent kinase 6 (CDK6), or both CDK4 and CDK6 (CDK4/6).

In another aspect, the invention provides a method of treating cancer in a subject comprising administering to the subject an effective amount of a cyclin dependent kinase (CDK) inhibitor and an effective amount of a bromodomain and extra-terminal domain (BET) inhibitor, wherein the CDK inhibitor is an inhibitor of cyclin dependent kinase 4 (CDK4), cyclin dependent kinase 6 (CDK6), or both CDK4 and CDK6 (CDK4/6).

In another aspect, the invention provides a method of treating cancer in a subject comprising administering to the subject an amount of a cyclin dependent kinase (CDK) inhibitor and an amount of a bromodomain and extra-terminal domain (BET) inhibitor, wherein the CDK inhibitor is an inhibitor of cyclin dependent kinase 4 (CDK4), cyclin dependent kinase 6 (CDK6), or both CDK4 and CDK6 (CDK4/6), and wherein the amounts of the CDK inhibitor and the BET inhibitor are together effective for the treatment of cancer.

In another aspect, the invention provides a method of treating cancer in a subject comprising administering to the subject a combination therapy comprising a CDK inhibitor and a BET inhibitor, wherein the CDK inhibitor is a CDK4 inhibitor, a CDK6 inhibitor, or a CDK4/6 inhibitor.

In another aspect, the invention provides a method of treating cancer in a subject comprising administering to the subject a combination therapy comprising an effective amount of a CDK inhibitor and an effective amount of a BET inhibitor, wherein the CDK inhibitor is a CDK4 inhibitor, a CDK6 inhibitor, or a CDK4/6 inhibitor.

In another aspect, the invention provides a method of treating cancer in a subject comprising administering to the subject a combination therapy comprising an amount of a CDK inhibitor and an amount of a BET inhibitor, wherein the CDK inhibitor is a CDK4 inhibitor, a CDK6 inhibitor, or a CDK4/6 inhibitor, and wherein the amounts of the CDK inhibitor and the BET inhibitor are together effective for the treatment of cancer. In another aspect, the invention provides a method of treating cancer in a subject comprising administering to the subject an effective amount of palbociclib or a pharmaceutically acceptable salt thereof and an effective amount of a bromodomain and extra-terminal domain (BET) inhibitor. In some embodiments of this aspect, the BET inhibitor is mivebresib or AZD5153, or a pharmaceutically acceptable salt thereof. In another aspect, the invention provides a method treating cancer in a subject comprising administering to the subject an effective amount of palbociclib or a pharmaceutically acceptable salt thereof and an effective amount of mivebresib, or a pharmaceutically acceptable salt thereof.

In another aspect, the invention provides a combination comprising a CDK inhibitor and a BET inhibitor, wherein the CDK inhibitor is a CDK4 inhibitor, a CDK6 inhibitor, or a CDK4/6 inhibitor.

In another aspect, the invention provides a combination comprising a CDK inhibitor and a BET inhibitor for use in the treatment of cancer in a subject, wherein the CDK inhibitor is a CDK4 inhibitor, a CDK6 inhibitor, or a CDK4/6 inhibitor.

In another aspect, the invention provides a combination comprising a CDK inhibitor and a BET inhibitor for use as a medicament, wherein the CDK inhibitor is a CDK4 inhibitor, a CDK6 inhibitor, or a CDK4/6 inhibitor.

In another aspect, the invention provides a synergistic combination comprising a CDK inhibitor and a BET inhibitor, wherein the CDK inhibitor is a CDK4 inhibitor, a CDK6 inhibitor, or a CDK4/6 inhibitor.

In another aspect, the invention provides a synergistic combination comprising a CDK inhibitor and a BET inhibitor for use in the treatment of cancer in a subject, wherein the CDK inhibitor is a CDK4 inhibitor, a CDK6 inhibitor, or a CDK4/6 inhibitor.

In another aspect, the invention provides a synergistic combination comprising a CDK inhibitor and a BET inhibitor for use as a medicament, wherein the CDK inhibitor is a CDK4 inhibitor, a CDK6 inhibitor, or a CDK4/6 inhibitor.

In another aspect, the invention provides a combination comprising palbociclib or a pharmaceutically acceptable salt thereof and a BET inhibitor. In some embodiments of this aspect, the BET inhibitor is mivebresib or AZD5153, or a pharmaceutically acceptable salt thereof. In specific embodiments of this aspect, the BET inhibitor is mivebresib or a pharmaceutically acceptable salt thereof. In some such embodiments, the combination is a synergistic combination.

In another aspect, the invention provides use of a CDK inhibitor and a BET inhibitor for the treatment of cancer in a subject, wherein the CDK inhibitor is a CDK4 inhibitor, a CDK6 inhibitor or a CDK4/6 inhibitor.

In another aspect, the invention provides use of an effective amount of a CDK inhibitor and an effective amount of a BET inhibitor for the treatment of cancer in a subject, wherein the CDK inhibitor is a CDK4 inhibitor, a CDK6 inhibitor or a CDK4/6 inhibitor.

In another aspect, the invention provides use of an amount of a CDK inhibitor and an amount of a BET inhibitor for the treatment of cancer in a subject, wherein the CDK inhibitor is a CDK4 inhibitor, a CDK6 inhibitor or a CDK4/6 inhibitor, and wherein the amounts of the CDK inhibitor and the BET inhibitor are together effective for the treatment of cancer.

In another aspect, the invention provides use of a combination comprising a CDK inhibitor and a BET inhibitor for the treatment of cancer in a subject, wherein the CDK inhibitor is a CDK4 inhibitor, a CDK6 inhibitor or a CDK4/6 inhibitor.

In another aspect, the invention provides use of palbociclib or a pharmaceutically acceptable salt thereof and a BET inhibitor for the treatment of cancer in a subject.

In another aspect, the invention provides use of an effective amount of palbociclib or a pharmaceutically acceptable salt thereof and an effective amount of a BET inhibitor for the treatment of cancer in a subject.

In another aspect, the invention provides use of an amount of palbociclib or a pharmaceutically acceptable salt thereof and an amount of a BET inhibitor for the treatment of cancer in a subject, wherein the amounts of the CDK inhibitor and the BET inhibitor are together effective for the treatment of cancer.

In another aspect, the invention provides use of a combination comprising palbociclib or a pharmaceutically acceptable salt thereof and a BET inhibitor for the treatment of cancer in a subject.

In some embodiments of each of the foregoing uses, the BET inhibitor is mivebresib or AZD5153, or a pharmaceutically acceptable salt thereof. In specific embodiments, the BET inhibitor is mivebresib or a pharmaceutically acceptable salt thereof.

In another aspect, the invention provides a pharmaceutical composition comprising a CDK inhibitor, a BET inhibitor, and a pharmaceutically acceptable carrier or excipient, wherein the CDK inhibitor is a CDK4 inhibitor, a CDK6 inhibitor, or a CDK4/6 inhibitor.

In another aspect, the invention provides a composition for use in the treatment of cancer comprising a CDK inhibitor and a BET inhibitor, wherein the CDK inhibitor is a CDK4 inhibitor, a CDK6 inhibitor, or a CDK4/6 inhibitor.

In another aspect, the invention provides a composition for use in the treatment of cancer comprising a CDK inhibitor, a BET inhibitor, and a pharmaceutically acceptable carrier or excipient, wherein the CDK inhibitor is a CDK4 inhibitor, a CDK6 inhibitor, or a CDK4/6 inhibitor.

In another aspect, the invention provides a pharmaceutical composition comprising palbociclib or a pharmaceutically acceptable salt thereof, a BET inhibitor, and a pharmaceutically acceptable carrier or excipient.

In another aspect, the invention provides a composition for use in the treatment of cancer comprising palbociclib or a pharmaceutically acceptable salt thereof and a BET inhibitor.

In another aspect, the invention provides a composition for use in the treatment of cancer comprising palbociclib or a pharmaceutically acceptable salt thereof, a BET inhibitor, and a pharmaceutically acceptable carrier or excipient.

In some embodiments of each of the foregoing compositions, the BET inhibitor is mivebresib or AZD5153, or a pharmaceutically acceptable salt thereof. In specific embodiments, the BET inhibitor is mivebresib or a pharmaceutically acceptable salt thereof.

In another aspect, the invention provides a kit which comprises a first container, a second container and a package insert, wherein the first container comprises at least one dose of a CDK inhibitor, wherein the CDK inhibitor is a CDK4 inhibitor, a CDK6 inhibitor or a CDK4/6 inhibitor; the second container comprises at least one dose of a BET inhibitor; and the package insert comprises instructions for treating cancer in a subject using the medicaments.

In another aspect, this invention relates to a CDK inhibitor for use in the treatment of cancer in a subject, wherein the CDK inhibitor is used in combination with a BET inhibitor, and wherein the CDK inhibitor is a CDK4 inhibitor, a CDK6 inhibitor or a CDK4/6 inhibitor.

In another aspect, this invention relates to a BET inhibitor for use in the treatment of cancer in a subject, wherein the BET inhibitor is used in combination with a CDK inhibitor, and wherein the CDK inhibitor is a CDK4 inhibitor, a CDK6 inhibitor or a CDK4/6 inhibitor.

In another aspect, the invention provides use of a CDK inhibitor and a BET inhibitor in the manufacture of a medicament for the treatment of cancer in a subject, wherein the CDK inhibitor is a CDK4 inhibitor, a CDK6 inhibitor or a CDK4/6 inhibitor.

In another aspect, the invention provides use of a CDK inhibitor for the manufacture of a medicament for the treatment of cancer, wherein the CDK inhibitor is a CDK4 inhibitor, a CDK6 inhibitor or a CDK4/6 inhibitor, and the medicament is adapted for use in combination with a BET inhibitor.

In another aspect, the invention provides use of a BET inhibitor for the manufacture of a medicament for the treatment of cancer, wherein the medicament is adapted for use in combination with a CDK inhibitor, and wherein the CDK inhibitor is a CDK4 inhibitor, a CDK6 inhibitor or a CDK4/6 inhibitor.

In another aspect, this invention relates to a pharmaceutical composition comprising a CDK inhibitor, wherein the CDK inhibitor is a CDK4 inhibitor, a CDK6 inhibitor or a CDK4/6 inhibitor, and a pharmaceutically acceptable carrier for use in the treatment of cancer in a subject, wherein the pharmaceutical composition comprising the CDK inhibitor is used in combination with a pharmaceutical composition comprising a BET inhibitor and a pharmaceutically acceptable carrier.

In another aspect, this invention relates to a pharmaceutical composition comprising a BET inhibitor and a pharmaceutically acceptable carrier for use in the treatment of cancer in a subject, wherein the pharmaceutical composition comprising the BET inhibitor is used in combination with a pharmaceutical composition comprising a CDK inhibitor and a pharmaceutically acceptable carrier, wherein the CDK inhibitor is a CDK4 inhibitor, a CDK6 inhibitor or a CDK4/6 inhibitor.

In some embodiments of each of the aspects herein, the CDK inhibitor is a CDK4/6 inhibitor. In some such embodiments, the CDK4/6 inhibitor is selected from the group consisting of palbociclib, ribociclib and abemaciclib, or a pharmaceutically acceptable salt thereof. In other such embodiments, the CDK4/6 inhibitor is selected from the group consisting of palbociclib, ribociclib, abemaciclib, G1T38, trilaciclib and SHR6390, or a pharmaceutically acceptable salt thereof. In specific embodiments, the CDK4/6 inhibitor is palbociclib, or a pharmaceutically acceptable salt thereof. In further embodiments, the CDK4/6 inhibitor is ribociclib or abemaciclib, or a pharmaceutically acceptable salt thereof.

In some embodiments of each of the aspects herein, the CDK inhibitor is a CDK4 inhibitor. In other embodiments of each of the aspects herein, the CDK inhibitor is a CDK6 inhibitor. In further embodiments of each of the foregoing aspects, the CDK inhibitor is a pan-CDK inhibitor that inhibits CDK4.

In some embodiments of each of the aspects herein, the BET inhibitor is a BRD4 inhibitor, a BRD2 inhibitor or a BRD2/4 inhibitor. In some such embodiments, the BET inhibitor further inhibits BRDT.

In some embodiments, the BET inhibitor is selected from the group consisting of: mivebresib; AZD5153; BAY1238097; BI-894999; BMS-986158; CPI-0610; FT-1101; GSK525762; GSK2820151; GS-5829; INCB054329; INCB057643; N-methyl-2-pyrrolidone; MK-8628; RO6870810; apabetalone; and ZEN003694; or a pharmaceutically acceptable salt thereof. In specific embodiments, the BET inhibitor is mivebresib or AZD5153, or a pharmaceutically acceptable salt thereof. In some such embodiments, the BET inhibitor is mivebresib, or a pharmaceutically acceptable salt thereof. In other such embodiments, the BET inhibitor is AZD5153, or a pharmaceutically acceptable salt thereof.

In each of the foregoing aspects and embodiments, the CDK inhibitor and the BET inhibitor may independently optionally be in the form of a pharmaceutically acceptable salt.

In preferred embodiments of each of the aspects described herein, the CDK inhibitor is palbociclib or a pharmaceutically acceptable salt thereof and the BET inhibitor is mivebresib or AZD5153, or a pharmaceutically acceptable salt thereof. In specific embodiments of each of the aspects described herein, the CDK inhibitor is palbociclib or a pharmaceutically acceptable salt thereof and the BET inhibitor is mivebresib or a pharmaceutically acceptable salt thereof. In specific embodiments of each of the aspects described herein, the CDK inhibitor is palbociclib or a pharmaceutically acceptable salt thereof and the BET inhibitor is AZD5153 or a pharmaceutically acceptable salt thereof.

In frequent embodiments of each of the aspects of the invention, the subject is a human.

Examples of cancers include, but are not limited to, carcinoma, lymphoma, leukaemia, blastoma, and sarcoma. In some embodiments the methods, uses and combinations of the present invention may be useful for the treatment of one or more cancers including but not limited to cancers of the:

circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue;

respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma);

bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germ inoma [pinealoma], glioblastoma multiform, oligodendroglioma, schwannoma, retinoblastoma, congenital tumors), spinal cord neurofibroma, meningioma, glioma, sarcoma);

reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs;

hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma];

oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx;

skin, for example, malignant melanoma, cutaneous melanoma, basal cell carcinoma (BCC), squamous cell carcinoma (SCC), Karposi's sarcoma, moles dysplastic nevi, lipoma, angioma, dermatofibroma, and keloids;

adrenal glands: neuroblastoma; and

other tissues including connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

More particular examples of cancer when used herein in connection with the present invention include cancers of the breast, ovary, lung (including SCLC and

NSCLC), skin, colon, bladder, liver, stomach, prostate, kidney, esophagus, nasopharynx, thyroid, cervix, pancreas, head and neck, or sarcomas, or a combination of one or more of the foregoing cancers.

Still more specifically, examples of breast cancer in connection with the present invention include: hormone receptor positive (HR+) breast cancer, i.e., estrogen receptor positive (ER+) and/or progesterone receptor positive (PR+); human epidermal growth factor receptor 2 negative (HER2−) breast cancer; human epidermal growth factor receptor 2 positive (HER2+) breast cancer; and triple negative breast cancer (TNBC). In one embodiment of the invention, the cancer is a solid tumor.

In some embodiments of each of the aspects described herein, the cancer is breast cancer or pancreatic cancer.

In frequent embodiments, the cancer is breast cancer. In some such embodiments, the cancer is HR+ breast cancer, including ER+ and/or PR+ breast cancer.

In further embodiments, the cancer is HER2− breast cancer. In frequent embodiments, the cancer is HR+ HER2− breast cancer.

In other embodiments, the cancer is HER2+breast cancer. In some such embodiments, the cancer is HR+ HER2+ breast cancer. In other embodiments, the cancer is HR− HER2+ breast cancer.

In some embodiments, the cancer is TNBC, (i.e., ER−, PR− and HER2−). In some such embodiments, the cancer is associated with the BRCA1 or BRCA2 gene.

In some embodiment of each of the aspects described herein, the cancer is locally advanced. In some embodiments of each of the aspects described herein, the cancer is metastatic. In other embodiments of each of the aspects described herein, the cancer is refractory.

In some embodiments of each of the aspects described herein, the cancer is resistant to treatment with a CDK inhibitor, e.g., the cancer is resistant to treatment with a CDK4 inhibitor, a CDK6 inhibitor, or a CDK4/6 inhibitor. In other such embodiments, the cancer is resistant to treatment with a BET inhibitor.

In further embodiments of each of the aspects described herein, the cancer is resistant to treatment with one or more standard of care agents. In some such embodiments, the cancer is breast cancer that is resistant to treatment with endocrine therapy, such as aromatase inhibitors, SERDs or SERMs. In other embodiments, the cancer is resistant to treatment with chemotherapeutic agents, including but not limited to platinum agents, taxanes, docetaxel or gemcitabine.

In another aspect, the invention provides a method of inhibiting cancer cell proliferation in a subject, comprising administering to the subject a combination therapy which comprises a CDK inhibitor and a BET inhibitor, wherein the CDK inhibitor is a CDK4 inhibitor, a CDK6 inhibitor or a CDK4/6 inhibitor, in an amount effective to inhibit cell proliferation.

In another aspect, the invention provides a method of inhibiting cancer cell invasiveness in a subject, comprising administering to the subject a combination therapy which comprises a CDK inhibitor and a BET inhibitor, wherein the CDK inhibitor is a CDK4 inhibitor, a CDK6 inhibitor or a CDK4/6 inhibitor, in an amount effective to inhibit cell invasiveness.

In another aspect, the invention provides a method of inducing apoptosis in cancer cells in a subject, comprising administering to the subject a combination therapy which comprises a CDK inhibitor and a BET inhibitor, wherein the CDK inhibitor is a CDK4 inhibitor, a CDK6 inhibitor or a CDK4/6 inhibitor, in an amount effective to induce apoptosis.

“Contacting” refers to bringing a compound or pharmaceutically acceptable salt of the invention and a cell expressing CDK4, CDK6 or CDK4/6 together in such a manner that the compound may affect the activity of CDK4, CDK6 or CDK4/6, either directly or indirectly. Contacting may be accomplished in vitro (i.e., in an artificial environment such as, e.g., without limitation, in a test tube or culture medium) or in vivo (i.e., within a living organism such as, without limitation, a mouse, rat or rabbit.)

In some embodiments, the cells are in a cell line, such as a cancer cell line. In other embodiments, the cells are in a tissue or tumor, and the tissue or tumor may be in a subject, including a human.

Dosage Forms and Regimens

Each therapeutic agent of the methods and combination therapies of the present invention may be administered either alone, or in a medicament (also referred to herein as a pharmaceutical composition) which comprises the therapeutic agent and one or more pharmaceutically acceptable carriers, excipients, or diluents, according to pharmaceutical practice.

As used herein, the term “combination therapy” refers to the administration of each therapeutic agent of the combination therapy of the invention, either alone or in a medicament, either sequentially, concurrently or simultaneously.

As used herein, the term “sequential” or “sequentially” refers to the administration of each therapeutic agent of the combination therapy of the invention, either alone or in a medicament, one after the other, wherein each therapeutic agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly.

As used herein, the term “concurrently” refers to the administration of each therapeutic agent in the combination therapy of the invention, either alone or in separate medicaments, wherein the second therapeutic agent is administered immediately after the first therapeutic agent, but that the therapeutic agents can be administered in any order. In a preferred embodiment the therapeutic agents are administered concurrently.

As used herein, the term “simultaneous” refers to the administration of each therapeutic agent of the combination therapy of the invention in the same medicament.

In some embodiments of the present invention, the CDK inhibitor and the BET inhibitor are administered sequentially, simultaneously or concurrently. In some such embodiments, the CDK inhibitor is administered before administration of the BET inhibitor. In other embodiments, the CDK inhibitor is administered after administration of the BET inhibitor. In other embodiments, the CDK inhibitor is administered concurrently with administration of the BET inhibitor. In further embodiments, the CDK inhibitor is administered simultaneously with the BET inhibitor. In each of the foregoing embodiments, it will be understood that the CDK inhibitor is a CDK4 inhibitor, a CDK6 inhibitor or a CDK4/6 inhibitor.

As will be understood by those skilled in the art, the combination therapy may be usefully administered to a subject during different stages of their treatment.

In some embodiments, the combination therapy is administered to a subject who is previously untreated, i.e. is treatment naïve.

In some embodiments, the combination therapy is administered to a subject who has failed to achieve a sustained response after a prior therapy with a biotherapeutic or chemotherapeutic agent, i.e. is treatment experienced.

The combination therapy may be administered prior to of following surgery to remove a tumor and/or may be used prior to, during or after radiation therapy, and/or may be used prior to, during or after chemotherapy.

The efficacy of combinations described herein in certain tumors may be enhanced by combination with other approved or experimental cancer therapies, e.g., radiation, surgery, chemotherapeutic agents, targeted therapies, agents that inhibit other signaling pathways that are dysregulated in tumors, and other immune enhancing agents, such as PD-1 or PD-L1 antagonists and the like. The methods, combinations and uses of the current invention may further comprise one or more additional anti-cancer agents.

Administration of combinations of the invention may be effected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a therapeutic agent of the combination therapy of the present invention may be administered as a single bolus, as several divided doses administered over time, or the dose may be proportionally reduced or increased as indicated by the exigencies of the therapeutic situation. It may be particularly advantageous to formulate a therapeutic agent in a dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention may be dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose may be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present invention. It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, taking into consideration factors such as the severity of the disorder or condition, the rate of administration, the disposition of the compound and the discretion of the prescribing physician. The dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

In some embodiments, at least one of the therapeutic agents in the combination therapy is administered using the same dosage regimen (dose, frequency and duration of treatment) that is typically employed when the agent is used as a monotherapy for treating the same cancer. In other embodiments, the subject received a lower total amount of at least one of the therapeutic agents in the combination therapy than when the same agent is used as a monotherapy, for example a lower dose of therapeutic agent, a reduced frequency of dosing and/or a shorter duration of dosing.

An effective dosage of a small molecule inhibitor is typically in the range of from about 0.001 to about 100 mg per kg body weight per day, preferably about 1 to about 35 mg/kg/day, in single or divided doses. For a 70 kg human, this would amount to about 0.01 to about 7 g/day, preferably about 0.02 to about 2.5 g/day, and more preferably from about 0.02 to about 1.0 g/day. In some instances, dosage levels at the lower limit of the aforesaid range may be more than adequate, while in other cases still larger doses may be employed without causing any harmful side effect, provided that such larger doses are first divided into several small doses for administration throughout the day.

In some embodiments, the CDK inhibitor, or a pharmaceutically acceptable salt or solvate thereof, is administered at a daily dosage of from about 50 mg to about 1000 mg per day, preferably from about 50 mg to about 600 mg per day, and more preferably from about 75 mg to about 200 mg per day. In certain embodiments, the CDK inhibitor is palbociclib, or a pharmaceutically acceptable salt or solvate thereof, which is administered at a daily dosage of 75 mg, 100 mg, or 125 mg per day. In other embodiments, the CDK inhibitor is ribociclib, or a pharmaceutically acceptable salt or solvate thereof, which is administered at a daily dosage of about 200 mg to about 600 mg per day; or abemaciclib, or a pharmaceutically acceptable salt or solvate thereof, which is administered at a daily dosage of about 150 mg or about 400 mg per day.

In some embodiments, the CDK inhibitor, or a pharmaceutically acceptable salt or solvate thereof, is administered at a daily dosage of about 50 mg, about 75 mg, about 100 mg, about 125 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, or about 1500 mg. This dosage may be administered as a single dose (q.d.), or optionally may be subdivided into smaller doses, suitable for b.i.d., t.i.d. or q.i.d. administration.

In some embodiments, the BET inhibitor, or a pharmaceutically acceptable salt or solvate thereof, is administered at a daily dosage of about 50 mg, about 75 mg, about 100 mg, about 125 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, or about 1500 mg. This dosage may be administered as a single dose (q.d.), or optionally may be subdivided into smaller doses, suitable for b.i.d., t.i.d. or q.i.d. administration.

Repetition of the administration or dosing regimens, or adjustment of the administration or dosing regimen may be conducted as necessary to achieve the desired treatment. An “intermittent dosing schedule” as used herein refers to an administration or dosing regimen that includes a period of dose interruption, e.g. days off treatment. Repetition of 14 or 21 day treatment cycles with a 7 day treatment interruption between the treatment cycles is an example of an intermittent dosing schedule. Such schedules, with 2 or 3 weeks on treatment and 1 week off treatment, are sometimes referred to as a 2/1-week or 3/1-week treatment cycle, respectively.

A “continuous dosing schedule” as used herein is an administration or dosing regimen without dose interruptions, e.g. without days off treatment. Repetition of 21 or 28 day treatment cycles without dose interruptions between the treatment cycles is an example of a continuous dosing schedule.

In some embodiments, the CDK inhibitor and the BET inhibitor are administered in an intermittent dosing schedule. In other embodiments, the CDK inhibitor and the BET inhibitor are administered in a continuous dosing schedule.

In still other embodiments, one of the CDK inhibitor and the BET inhibitor is administered in an intermittent dosing schedule (e.g., a 2/1-week or 3/1-week schedule) and the other is administered in a continuous dosing schedule. In some such embodiments, the CDK inhibitor is administered in an intermittent dosing schedule and the BET inhibitor is administered in a continuous dosing schedule. In other such embodiments, the CDK inhibitor is administered in a continuous dosing schedule and the BET inhibitor is administered in an intermittent dosing schedule.

In some embodiments of the present invention, the CDK inhibitor and the BET inhibitor are dosed in amounts which together are effective in treating the cancer.

In some embodiments of the present invention, the CDK inhibitor and the BET inhibitor are dosed in amounts which together are synergistic.

In some embodiments of the present invention, the CDK inhibitor and the BET inhibitor are dosed in amounts which together are additive.

In each of the foregoing embodiments, it will be understood that the CDK inhibitor is a CDK4 inhibitor, a CDK6 inhibitor or a CDK4/6 inhibitor.

### Pharmaceutical Compositions and Routes of Administration

A “pharmaceutical composition” refers to a mixture of one or more of the therapeutic agents described herein, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients.

As used herein, a “pharmaceutically acceptable carrier” refers to a carrier or diluent that does not cause significant irritation to an organism and does not abrogate the biological activity and properties of the active compound or therapeutic agent.

The pharmaceutical acceptable carrier may comprise any conventional pharmaceutical carrier or excipient. The choice of carrier and/or excipient will to a large extent depend on factors such as the particular mode of administration, the effect of the excipient on solubility and stability, and the nature of the dosage form.

Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulation, solution or suspension, for parenteral injection as a sterile solution, suspension or emulsion, for topical administration as an ointment or cream, or for rectal administration as a suppository.

Exemplary parenteral administration forms include solutions or suspensions of an active compound in a sterile aqueous solution, for example, aqueous propylene glycol or dextrose solutions. Such dosage forms may be suitably buffered, if desired.

The pharmaceutical composition may be in unit dosage forms suitable for single administration of precise amounts.

Pharmaceutical compositions suitable for the delivery of the therapeutic agents of the combination therapies of the present invention, and methods for their preparation will be readily apparent to those skilled in the art. Such compositions and methods for their preparation may be found, for example, in ‘Remington's Pharmaceutical Sciences’, 19th Edition (Mack Publishing Company, 1995), the disclosure of which is incorporated herein by reference in its entirety.

Therapeutic agents of the combination therapies of the invention may be administered orally. Oral administration may involve swallowing, so that the therapeutic agent enters the gastrointestinal tract, or buccal or sublingual administration may be employed by which the therapeutic agent enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Therapeutic agents of the combination therapies of the present invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, the therapeutic agent may make up from 1 wt % to 80 wt % of the dosage form, more typically from 5 wt % to 60 wt % of the dosage form. In addition to the active agent, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant may comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste-masking agents.

Exemplary tablets may contain up to about 80 wt % active agent, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid formulations for oral administration may be formulated to be immediate and/or modified release. Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles may be found in Verma et al, Pharmaceutical Technology On-line, 25(2), 1-14 (2001). The use of chewing gum to achieve controlled release is described in WO 00/35298. The disclosures of these references are incorporated herein by reference in their entireties.

In one embodiment, a pharmaceutical composition useful for the combination therapy of the present invention comprises only a single therapeutic agent, for example either a CDK inhibitor or a BET inhibitor.

In another embodiment, a pharmaceutical composition useful for the combination therapy of the present invention comprises both a CDK inhibitor and a BET inhibitor.

The therapeutic agents of the combination therapies of the present invention may conveniently be combined in the form of a kit suitable for coadministration of the compositions.

In one aspect, the present invention relates to a kit which comprises a first container, a second container and a package insert, wherein the first container comprises at least one dose of a CDK inhibitor, wherein the CDK inhibitor is a CDK4 inhibitor, a CDK6 inhibitor or a CDK4/6 inhibitor; the second container comprises at least one dose of a BET inhibitor; and the package insert comprises instructions for treating a subject for cancer using the medicaments.

In one embodiment, the kit of the present invention may comprise one or both of the active agents in the form of a pharmaceutical composition, which pharmaceutical composition comprises an active agent, or a pharmaceutically acceptable salt or solvate thereof, and a pharmaceutically acceptable carrier. The kit may contain means for separately retaining said compositions, such as a container, divided bottle, or divided foil packet. An example of such a kit is the familiar blister pack used for the packaging of tablets, capsules and the like.

The kit may be particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid. The kit may further comprise other materials that may be useful in administering the medicaments, such as diluents, filters, IV bags and lines, needles and syringes, and the like.

### Further Therapeutic Agents

In a further aspect, the methods and combination therapies of the present invention may additionally comprise administering a further anti-cancer agents, such as anti-tumor agents, anti-angiogenesis agents, signal transduction inhibitors and antiproliferative agents, which amounts are together effective in treating said cancer. In some such embodiments, the anti-tumor agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and the like.

In one embodiment of the methods and combination therapies of the present invention, the regimen includes a further active agent, wherein the further active agent is an endocrine agent, such as an aromatase inhibitor, a SERD or a SERM.

Examples of anti-angiogenesis agents include for example VEGF inhibitors, VEGFR inhibitors, TIE-2 inhibitors, PDGFR inhibitors, angiopoetin inhibitors, PKCp inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent™) bevacizumab (Avastin™), axitinib (AG 13736), SU 14813 (Pfizer), and AG 13958 (Pfizer). Additional anti-angiogenesis agents include vatalanib (CGP 79787), Sorafenib (Nexavar™), pegaptanib octasodium (Macugen™), vandetanib (Zactima™), PF-0337210 (Pfizer), SU 14843 (Pfizer), AZD 2171 (AstraZeneca), ranibizumab (Lucentis™), Neovastat™ (AE 941), tetrathiomolybdata (Coprexa™), AMG 706 (Amgen), VEGF Trap (AVE 0005), CEP 7055 (Sanofi-Aventis), XL 880 (Exelixis), telatinib (BAY 57-9352), and CP-868,596 (Pfizer). Other anti-angiogenesis agents include enzastaurin (LY 317615), midostaurin (CGP 41251), perifosine (KRX 0401), teprenone (Selbex™) and UCN 01 (Kyowa Hakko). Other examples of anti-angiogenesis agents include celecoxib (Celebrex™), parecoxib (Dynastat™), deracoxib (SC 59046), lumiracoxib (Preige™), valdecoxib (Bextra™), rofecoxib (Vioxx™) iguratimod (Careram™), IP 751 (Invedus), SC-58125 (Pharmacia) and etoricoxib (Arcoxia™). Yet further anti-angiogenesis agents include exisulind (Aptosyn™) salsalate (Amigesic™), diflunisal (Dolobid™), ibuprofen (Motrin™), ketoprofen (Orudis™), nabumetone (Relafen™), piroxicam (Feldene™), naproxen (Aleve™, Naprosyn™), diclofenac (Voltaren™), indomethacin (Indocin™), sulindac (Clinoril™) tolmetin (Tolectin™), etodolac (Lodine™), ketorolac (Toradol™), and oxaprozin (Daypro™). Yet further anti-angiogenesis agents include ABT 510 (Abbott), apratastat (TMI 005), AZD 8955 (AstraZeneca), incyclinide (Metastat™), and PCK 3145 (Procyon). Yet further anti-angiogenesis agents include acitretin (Neotigason™) plitidepsin (aplidine™), cilengtide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™), thalidomide (Thalomid™), Ukrain™ (NSC 631570), Vitaxin™ (MEDI 522), and zoledronic acid (Zometa™)

In another embodiment the anti-cancer agent is a so called signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, IGF1R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WNT pathway and so called multi-targeted kinase inhibitors. Preferred signal transduction inhibitors include gefitinib (Iressa™), cetuximab (Erbitux™), erlotinib (Tarceva™), trastuzumab (Herceptin™) sunitinib (Sutent™), imatinib (Gleevec™), and PD325901 (Pfizer). Additional examples of signal transduction inhibitors which may be used in conjunction with a compound of the invention and pharmaceutical compositions described herein include BMS 214662 (Bristol-Myers Squibb), lonafarnib (Sarasar™), pelitrexol (AG 2037), matuzumab (EMD 7200), nimotuzumab (TheraCIM h-R3™), panitumumab (Vectibix™), Vandetanib (Zactima™), pazopanib (SB 786034), ALT 110 (Alteris Therapeutics), BIBW 2992 (Boehringer Ingelheim),and Cervene™ (TP 38). Other examples of signal transduction inhibitor include PF-2341066 (Pfizer), PF-299804 (Pfizer), canertinib (CI 1033), pertuzumab (Omnitarg™), Lapatinib (Tycerb™), pelitinib (EKB 569), miltefosine (Miltefosin™), BMS 599626 (Bristol-Myers Squibb), Lapuleucel-T (Neuvenge™) NeuVax™ (E75 cancer vaccine), Osidem™ (IDM 1), mubritinib (TAK-165), CP-724,714 (Pfizer), panitumumab (Vectibix™), lapatinib (Tycerb™), PF-299804 (Pfizer), pelitinib (EKB 569), and pertuzumab (Omnitarg™). Other examples of signal transduction inhibitors include ARRY 142886 (Array Biopharm), everolimus (Certican™), zotarolimus (Endeavor™), temsirolimus (Torisel™), AP 23573 (ARIAD), and VX 680 (Vertex). Additionally, other signal transduction inhibitors include XL 647 (Exelixis), sorafenib (Nexavar™), LE-AON (Georgetown University), and GI-4000 (Globelmmune). Other signal transduction inhibitors include ABT 751 (Abbott), alvocidib (flavopiridol), BMS 387032 (Bristol Myers), EM 1421 (Erimos), indisulam (E 7070), seliciclib (CYC 200), BIO 112 (Onc Bio), BMS 387032 (Bristol-Myers Squibb), PD 0332991 (Pfizer), and AG 024322 (Pfizer).

In another embodiment the anti-cancer agent is a so called classical antineoplastic agent. Classical antineoplastic agents include but are not limited to hormonal modulators such as hormonal, anti-hormonal, androgen agonist, androgen antagonist and anti-estrogen therapeutic agents, histone deacetylase (HDAC) inhibitors, gene silencing agents or gene activating agents, ribonucleases, proteosomics, Topoisomerase I inhibitors, Camptothecin derivatives, Topoisomerase II inhibitors, alkylating agents, antimetabolites, poly(ADP-ribose) polymerase-1 (PARP-1) inhibitor, microtubulin inhibitors, antibiotics, plant derived spindle inhibitors, platinum-coordinated compounds, gene therapeutic agents, antisense oligonucleotides, vascular targeting agents (VTAs), and statins. Examples of classical antineoplastic agents used in combination therapy with a compound of the invention, optionally with one or more other agents include, but are not limited to, glucocorticoids, such as dexamethasone, prednisone, prednisolone, methylprednisolone, hydrocortisone, and progestins such as medroxyprogesterone, megestrol acetate (Megace), mifepristone (RU-486), Selective Estrogen Receptor Modulators (SERMs; such as tamoxifen, raloxifene, lasofoxifene, afimoxifene, arzoxifene, bazedoxifene, fispemifene, ormeloxifene, ospemifene, tesmilifene, toremifene, trilostane and CHF 4227 (Cheisi), Selective Estrogen-Receptor Downregulators (SERD's; such as fulvestrant), exemestane (Aromasin), anastrozole (Arimidex), atamestane, fadrozole, letrozole (Femara), gonadotropin-releasing hormone (GnRH; also commonly referred to as luteinizing hormone-releasing hormone [LHRH]) agonists such as buserelin (Suprefact), goserelin (Zoladex), leuprorelin (Lupron), and triptorelin (Trelstar), abarelix (Plenaxis), bicalutamide (Casodex), cyproterone, flutamide (Eulexin), megestrol, nilutamide (Nilandron), and osaterone, dutasteride, epristeride, finasteride, Serenoa repens, PHL 00801, abarelix, goserelin, leuprorelin, triptorelin, bicalutamide, tamoxifen, exemestane, anastrozole, fadrozole, formestane, letrozole, and combinations thereof. Other examples of classical antineoplastic agents used in combination with a compound of the invention include but are not limited to suberolanilide hydroxamic acid (SAHA, Merck Inc./Aton Pharmaceuticals), depsipeptide (FR901228 or FK228), G2M-777, MS-275, pivaloyloxymethyl butyrate and PXD-101; Onconase (ranpirnase), PS-341 (MLN-341), Velcade (bortezomib), 9-aminocamptothecin, belotecan, BN-80915 (Roche), camptothecin, diflomotecan, edotecarin, exatecan (Daiichi), gimatecan, 10-hydroxycamptothecin, irinotecan HCl (Cam ptosar), lurtotecan, Orathecin (rubitecan, Supergen), SN-38, topotecan, camptothecin, 10-hydroxycamptothecin, 9-am inocamptothecin, irinotecan, SN-38, edotecarin, topotecan, aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide, AMD-473, altretamine, AP-5280, apaziquone, brostallicin, bendamustine, busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin, Eloxatin (oxaliplatin, Sanofi), streptozocin, satrplatin, and combinations thereof.

In another embodiment the anti-cancer agent is a so called dihydrofolate reductase inhibitors (such as methotrexate and NeuTrexin (trimetresate glucuronate)), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda™), cytosine arabinoside, Gemzar™ (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, N-(5-[N-(3,4-dihydro-2-methyl-4-oxoquinazolin-6-ylmethyl)-N-methylamino]-2-thenoyl)-L-glutamic acid, AG-014699 (Pfizer Inc.), ABT-472 (Abbott Laboratories), INO-1001 (Inotek Pharmaceuticals), KU-0687 (KuDOS Pharmaceuticals) and GPI 18180 (Guilford Pharm Inc) and combinations thereof.

Other examples of classical antineoplastic cytotoxic agents include, but are not limited to, Abraxane (Abraxis BioScience, Inc.), Batabulin (Amgen), EPO 906 (Novartis), Vinflunine (Bristol-Myers Squibb Company), actinomycin D, bleomycin, mitomycin C, neocarzinostatin (Zinostatin), vinblastine, vincristine, vindesine, vinorelbine (Navelbine), docetaxel (Taxotere), Ortataxel, paclitaxel (including Taxoprexin a DHA/paciltaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta™), DMXAA (Antisoma), ibandronic acid, L-asparaginase, pegaspargase (Oncaspar™), Efaproxiral (Efaproxyn™—radiation therapy), bexarotene (Targretin™), Tesmilifene (DPPE—enhances efficacy of cytotoxics), Theratope™ (Biomira), Tretinoin (Vesanoid™), tirapazamine (Trizaone™), motexafin gadolinium (Xcytrin™) Cotara™ (mAb), and NBI-3001 (Protox Therapeutics), polyglutamate-paclitaxel (Xyotax™) and combinations thereof. Further examples of classical antineoplastic agents include, but are not limited to, as Advexin (ING 201), TNFerade (GeneVec, a compound which express TNFalpha in response to radiotherapy), RB94 (Baylor College of Medicine), Genasense (Oblimersen, Genta), Combretastatin A4P (CA4P), Oxi-4503, AVE-8062, ZD-6126, TZT-1027, Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin, Niacin (Advicor, Kos Pharmaceuticals), Caduet, Lipitor, torcetrapib, and combinations thereof.

These and other aspects of the invention, including the exemplary specific embodiments listed below, will be apparent from the teachings contained herein.

### EXAMPLES

### Example 1—Preparation of BET Inhibitors

The BET Inhibitors B1 to B10 shown in Table 1 were prepared according to published procedures and used in the combination experiments.

TABLE 1
BET Inhibitors

| Test | | BRD4 IC₅₀ | |
|---|---|---|---|
| Compound | Structure | (nM) | Literature Ref. |
| B1 | 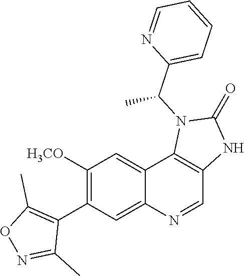 [SMILES: COc1cc2c(cc1-c1c(C)noc1C)ncc1[nH]c(=O)n([C@H](C)c3ccccn3)c12] | 25 | IBET-151 (GSK1210151) Mirguet et al., Bioorg. Med. Chem. Lett. (2012) 22(8): 2963-2967 |
| B2 | 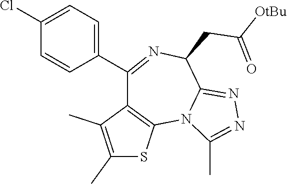 [SMILES: Cc1sc2c(c1C)C(c1ccc(Cl)cc1)=N[C@@H](CC(=O)OC(C)(C)C)c1nnc(C)n1-2] | 33 | JQ1 Filippakopoulos et al., Nature (2010) 468: 1067- 73 |
| B3 | 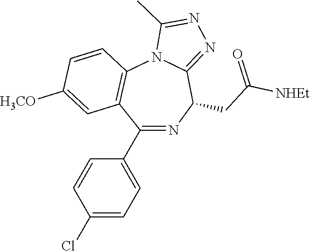 | 82 | IBET-762 (GSK525762) Mirguet et al., J. Med. Chem. (2013), 56: 7501-7515 |
| B4 | 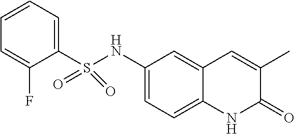 [SMILES: Cc1cc2cc(NS(=O)(=O)c3ccccc3F)ccc2[nH]c1=O] | 96 | PFI-2 WO 2013027168 |
| B5 | 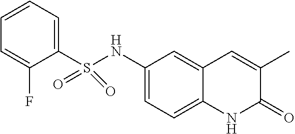 [SMILES: Cc1cc2cc(NS(=O)(=O)c3ccccc3F)ccc2[nH]c1=O] | 136 | PFI-1 Fish et al. J. Med. Chem. (2012) 55: 9831-9837 |
| B6 | 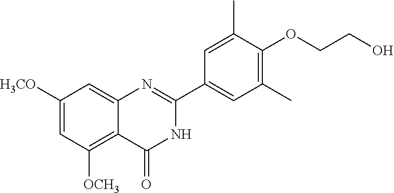 [SMILES: COc1cc(OC)c2c(=O)[nH]c(-c3cc(C)c(OCCO)c(C)c3)nc2c1] | 4000 | RVX-208 (RVX000222 or apabetalone) McClure et al., PloS One (2013) 8: 83190; U.S. Pat. No. 8,114,995 |
| B7 | 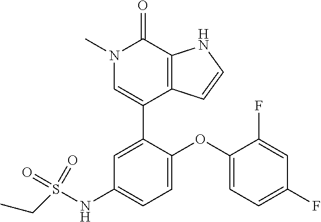 [SMILES: CCS(=O)(=O)Nc1ccc(Oc2ccc(F)cc2F)c(-c2cn(C)c(=O)c3[nH]ccc23)c1] | 1.5 | ABBV-075 (mivebresib) McDaniel et al., J. Med. Chem. (2017) 60: 8369- 8384 |
| B8 | 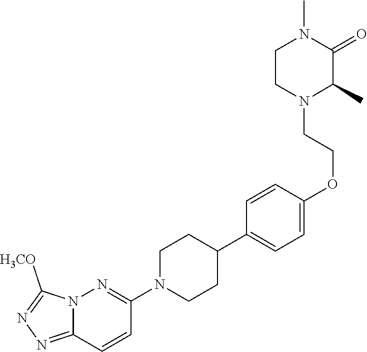 [SMILES: COc1nnc2ccc(N3CCC(c4ccc(OCCN5CCN(C)C(=O)[C@H]5C)cc4)CC3)nn12] | 5 | AZD5153 Bradbury et al., J. Med. Chem. (2016) 59: 7801- 7817 |
| B9 | 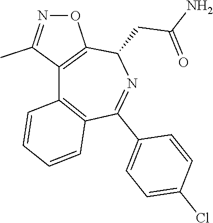 [SMILES: Cc1noc2c1-c1ccccc1C(c1ccc(Cl)cc1)=N[C@H]2CC(N)=O] | 39 | CPI-0610 Albrecht et al. J. Med. Chem. (2016) 59: 1330- 1339 |
| B10 | 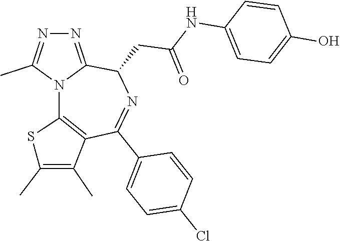 [SMILES: Cc1sc2c(c1C)C(c1ccc(Cl)cc1)=N[C@@H](CC(=O)Nc1ccc(O)cc1)c1nnc(C)n1-2] | 92 | MK-8628 (OTX015) U.S. Pat. No. 8,476,260 |

### Example 2—In Vitro Screen inT47D Breast Cancer Multicellular Tumor Spheroids

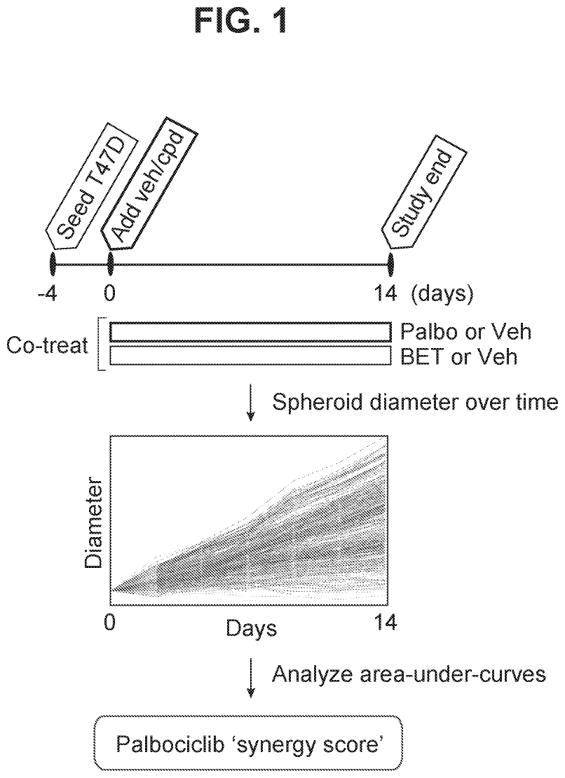

T47D multicellular tumor spheroids were grown in a low-adhesion 96-well plate for use in screening. A schematic representation is provided in FIG. 1. T47D cells were cultured in RPMI 1640 medium supplemented with 10% fetal bovine serum (GIBCO™). Two hundred T47D cells were seeded per well of 96-well ultralow attachment plates (ULA-96U, Thermo Fisher). Cells were grown for 4 days to allow spheroid formation, followed by initiation of compound treatment as indicated (day 0). Cells were treated either with vehicle (DMSO), or with 30 nM, 300 nM or 3000 nM of test compounds B1 to B6. At the same time, either vehicle (DMSO) or palbociclib (25 nM final concentration) was added to each sample. The medium was replaced every 3 days. Spheroid average diameter was quantified every 3 days (Celigo 200-BFFL-S, Nexcelcom) and spheroid growth curves were obtained. The area-under-curve (AUC) was quantified and used to calculate synergy scores for BET inhibitors in combination with palbociclib (Chalice Analyzer, Horizon Discovery). Synergy scores for test compounds in combination with 25 nM palbociclib are provided in Table 2.

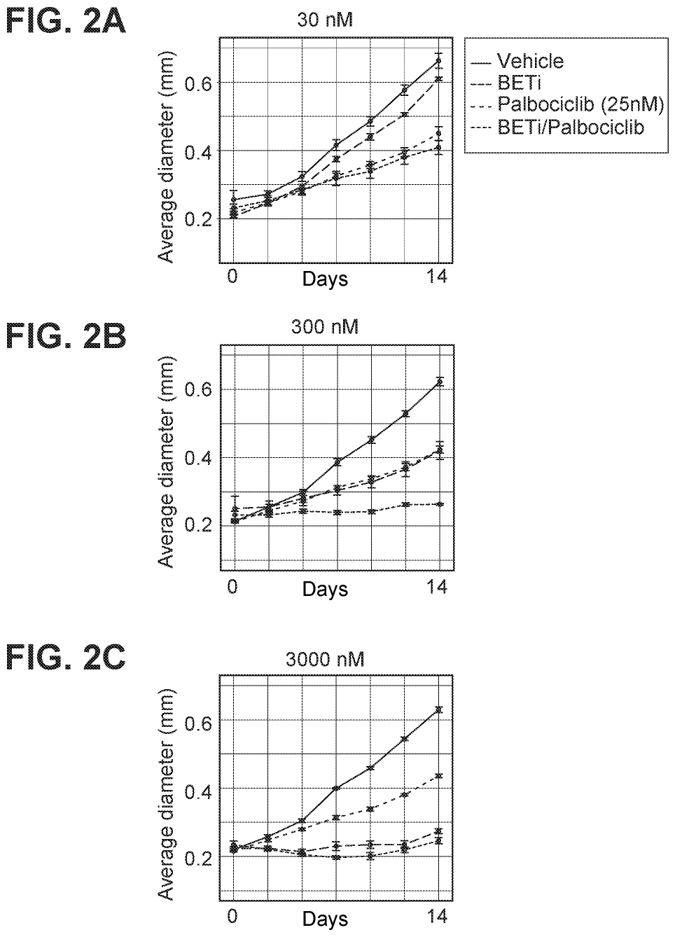

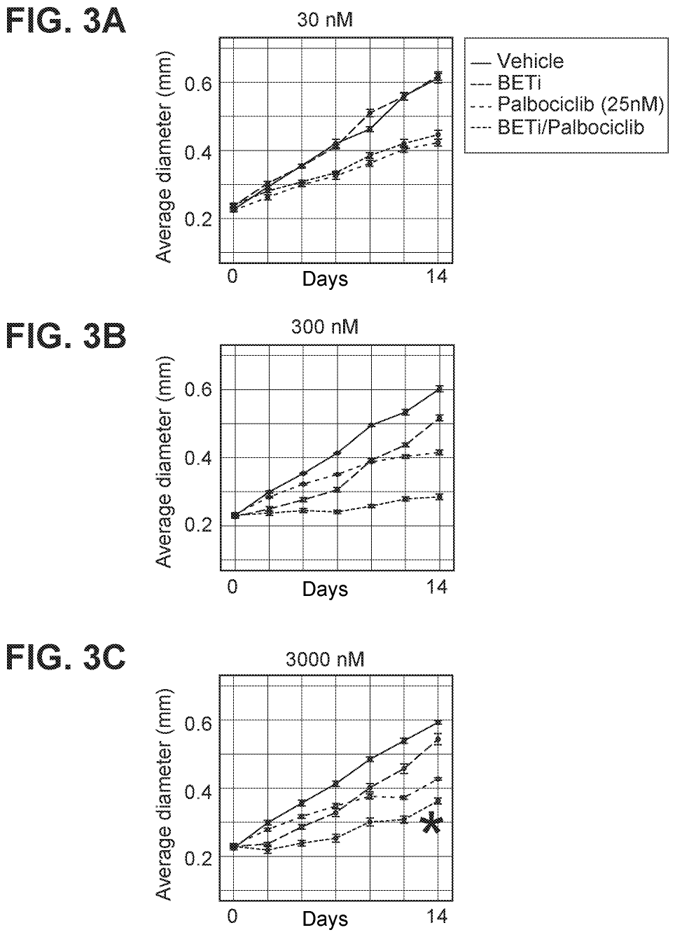

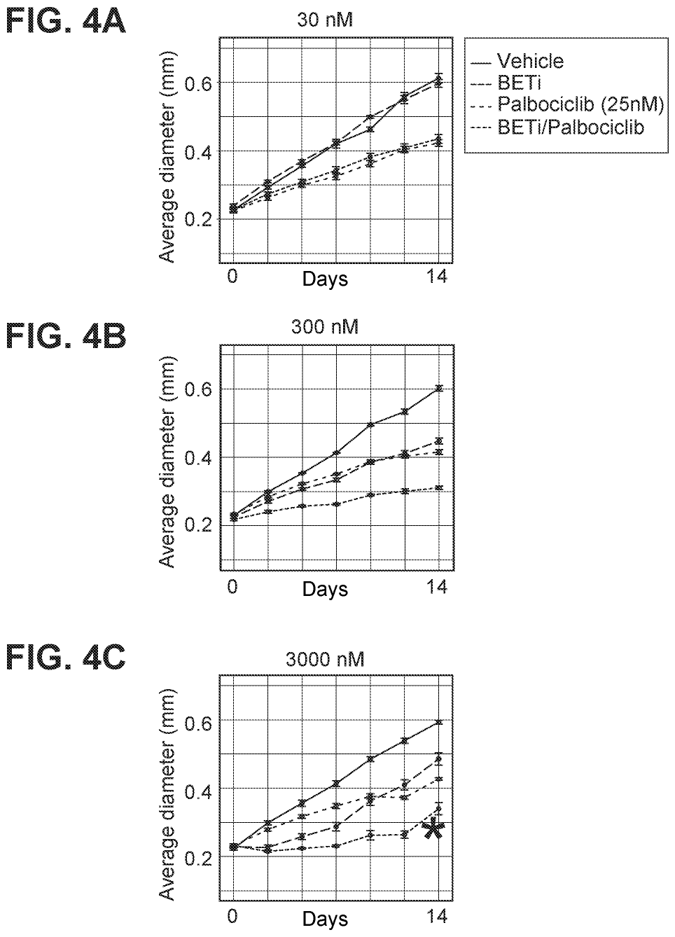

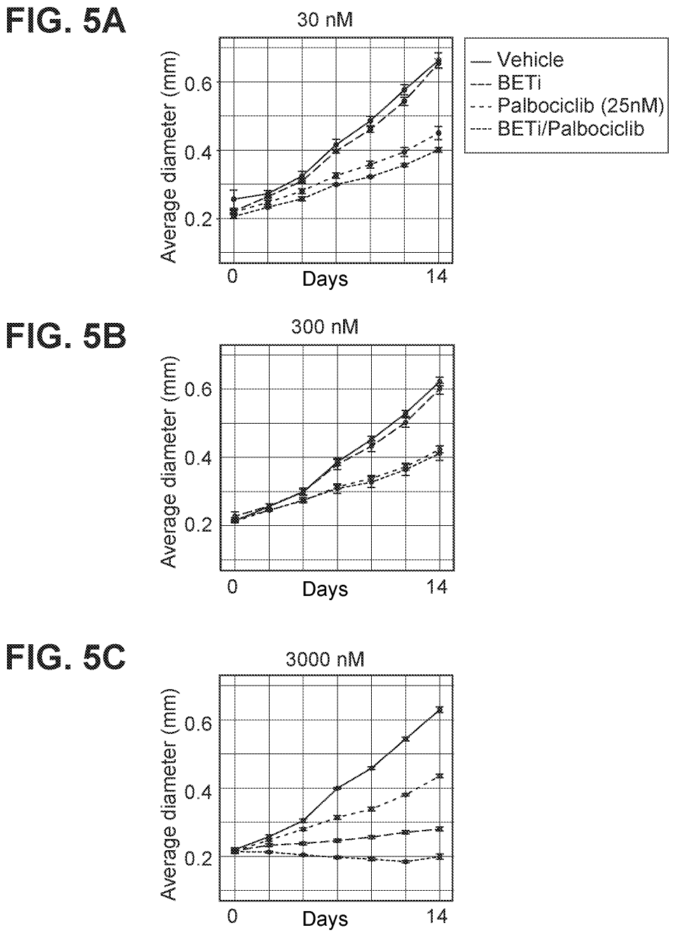

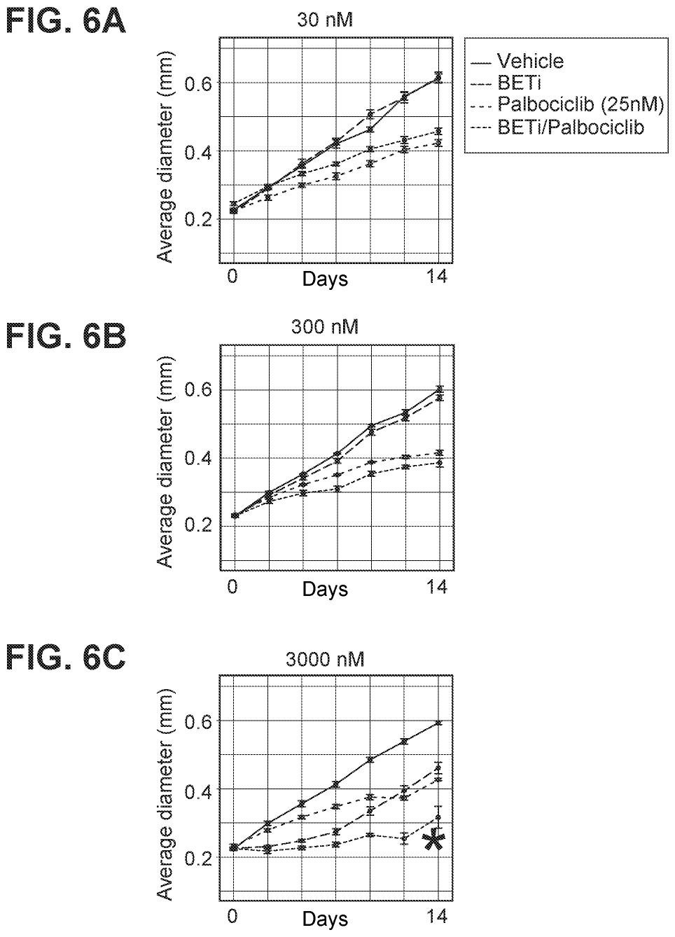

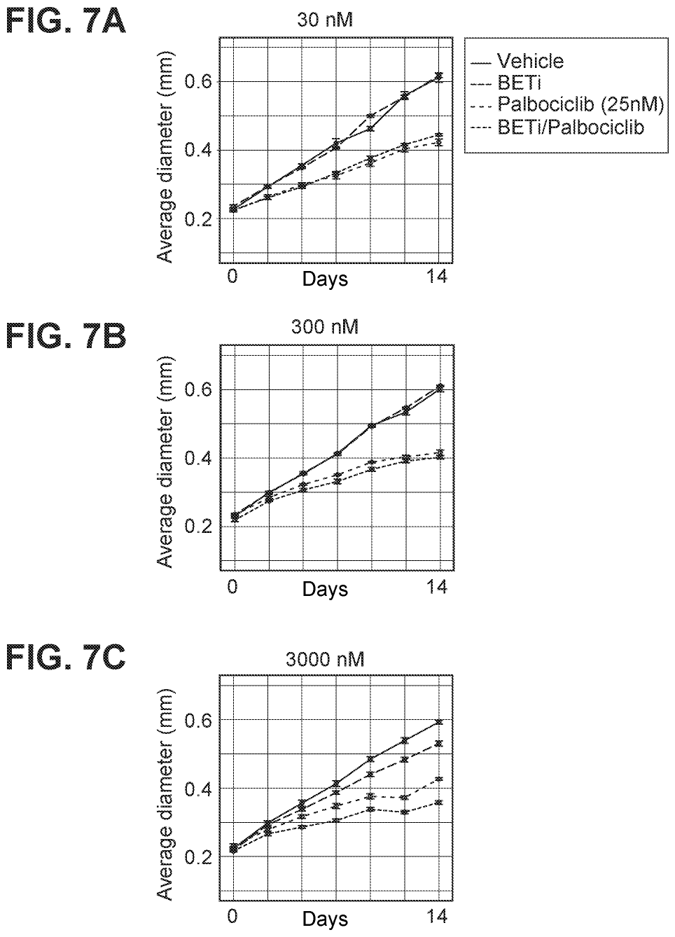

Original T47D spheroid growth curves for BET inhibitors B1 to B6 are shown in FIGS. 2A-2C (B1), FIGS. 3A-3C (B2), FIGS. 4A-4C (B3), FIGS. 5A-5C (B4), FIGS. 6A-6C (B5) and FIGS. 7A-7C (B6).

TABLE 2

| Test BET | Alternative | |
|---|---|---|
| inhibitor | name | Synergy score |
| B1 | I-BET151 | 1.09 |
| B2 | JQ1 | 1.08 |
| B3 | I-BET762 | 0.99 |
| B4 | PFI-1 | 0.79 |
| B5 | PFI-2 | 0.76 |
| B6 | RVX-208 | 0.33 |

### Example 3—In Vitro Screen inT47D Breast Cancer Multicellular Tumor Spheroids

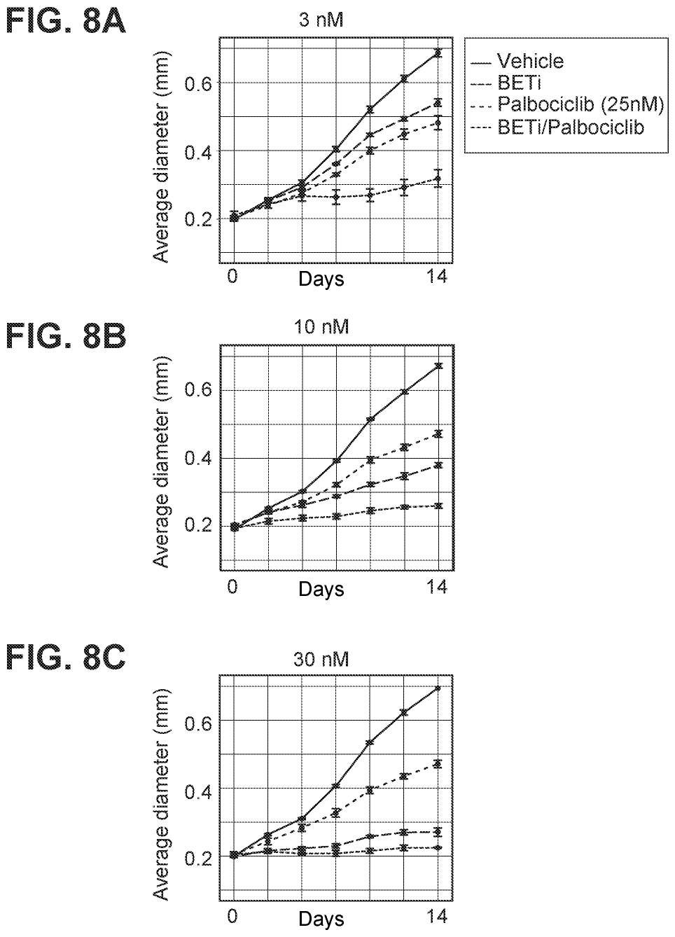

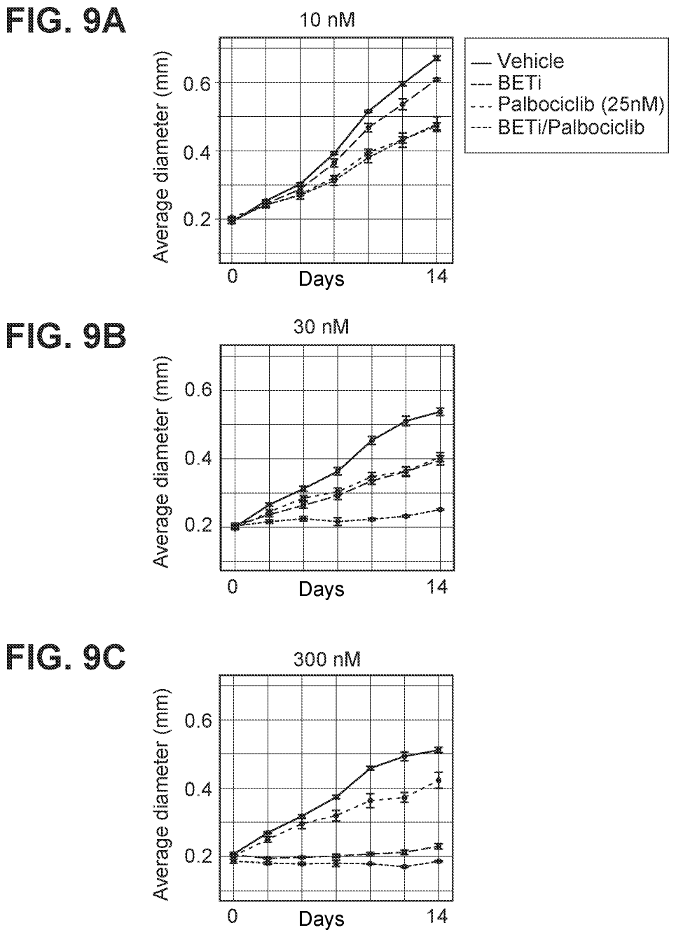

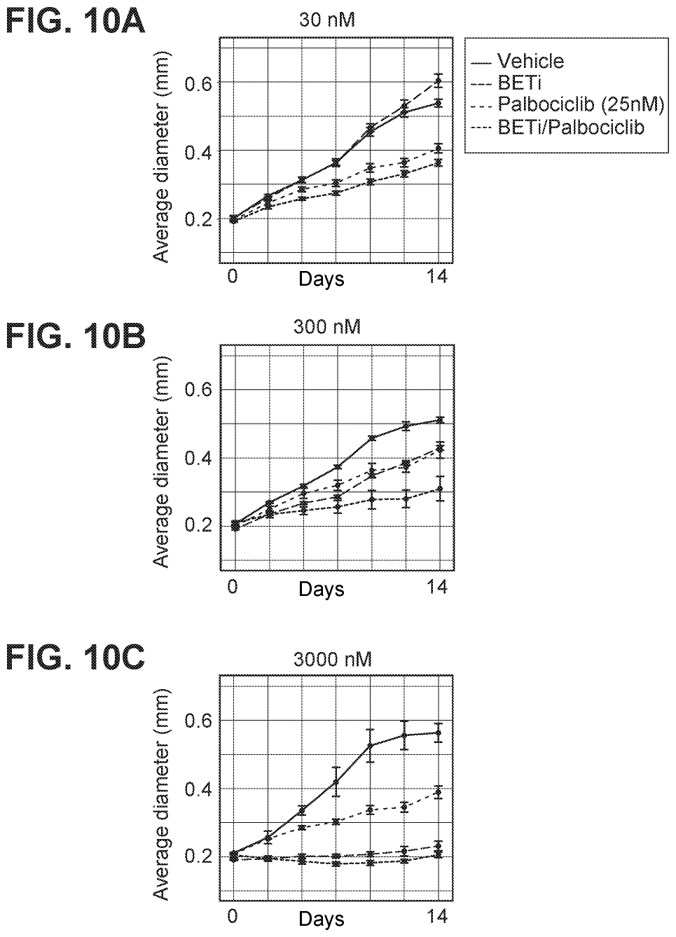

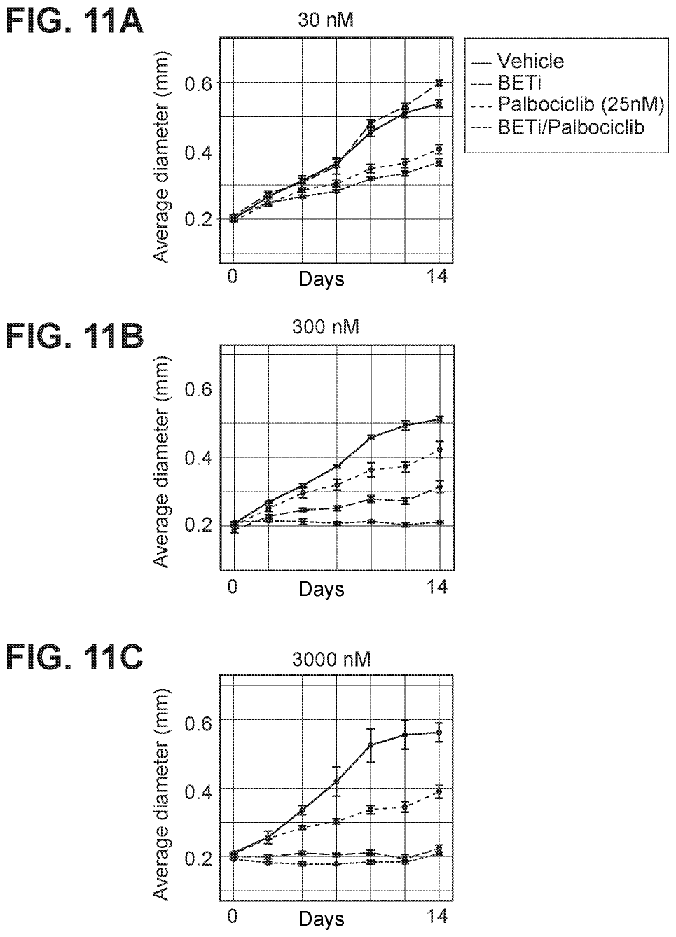

T47D multicellular tumor spheroids were grown as described in Example 2. Cells were treated with vehicle (DMSO) or with BET inhibitors B7, B8, B9 and B10 at appropriate concentrations depending on inhibitory potency (3 nM, 10 nM, 30 nM, 300 nM or 3000 nM). Either vehicle (DMSO) or palbociclib (25 nM final concentration) was added to each sample. T47D spheroid growth curves were generated as described in Example 2. Growth curves at the BET concentrations indicated are shown in FIGS. 8A-8C (B7); FIGS. 9A-9C (B8); FIGS. 10A-10C (B9); and FIGS. 11A-11C (B10).

### Example 4—In Vitro Screen in Hs766T PDAC Multicellular Tumor Spheroids

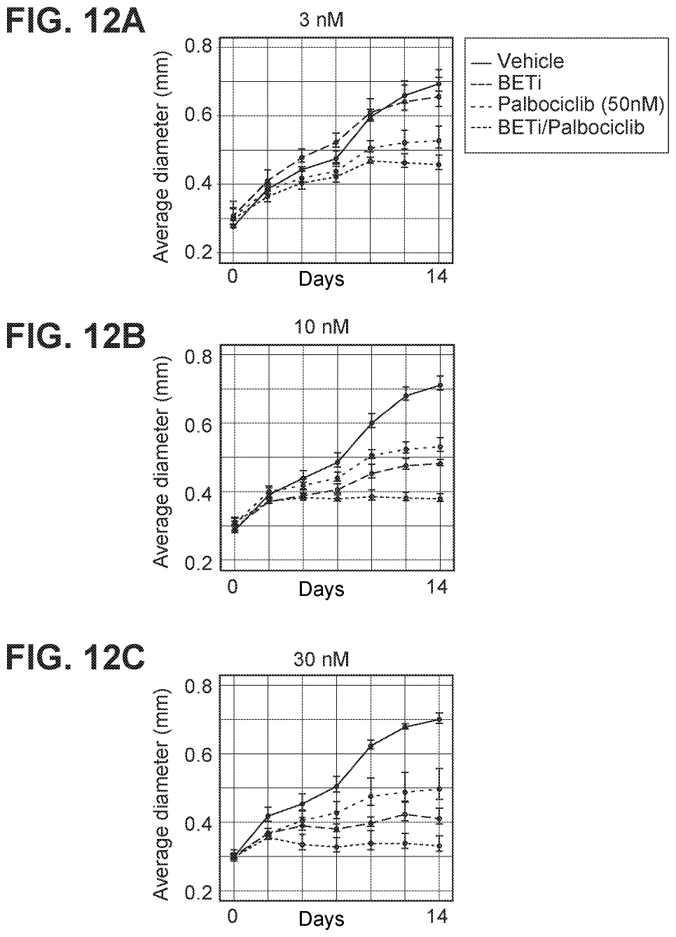

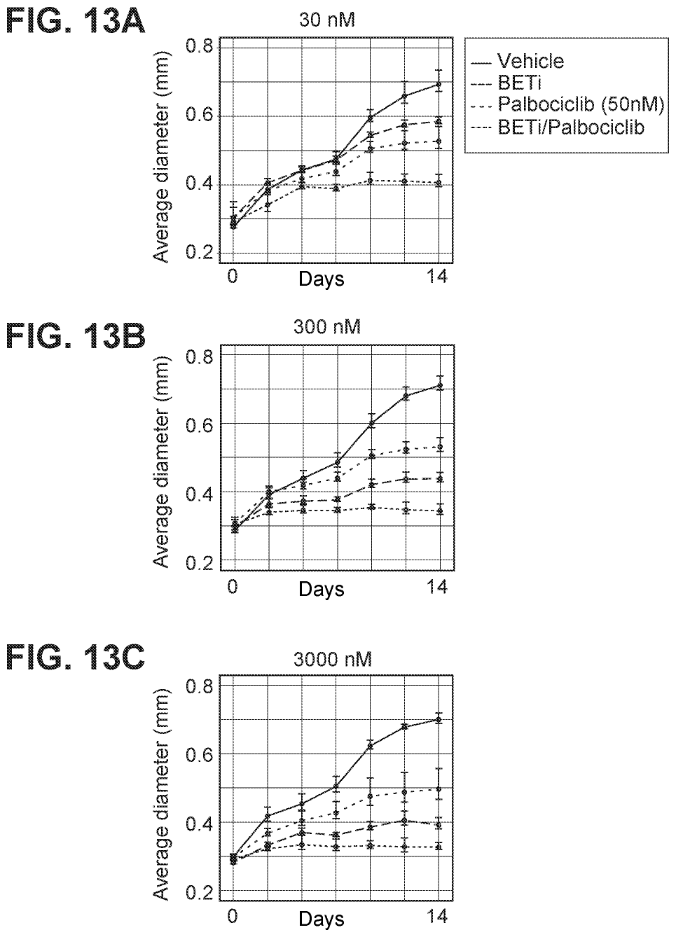

Hs766T multicellular tumor spheroids were grown in a low-adhesion 96-well plate for use in screening. Hs766T cells were cultured in DMEM medium supplemented with 10% fetal bovine serum (GIBCO™). 2% Matrigel (Cultrex, cat#3432-005-1) was added to the medium, and 400 cells/well were subsequently seeded per well of 96-well ultralow attachment plates (ULA-96U, Thermo Fisher). Cells were grown for 4 days to allow spheroid formation, followed by initiation of compound treatment as indicated (day 0). Cells were treated either with vehicle (DMSO), or with 3 nM, 10 nM or 30 nM of test compound B7, or 30 nM, 300 nM or 3000 nM of test compound B8. Either vehicle (DMSO) or palbociclib (50 nM final concentration) was added to each sample. The medium was replaced every 3 days. Spheroid average diameter was quantified every 3 days (Celigo 200-BFFL-S, Nexcelcom). Spheroid growth curves, generated as described in Example 2, are shown in FIGS. 12A-12C (B7); and FIGS. 13A-13C (B8).

### Example 5—In Vivo Study in MCF-7 Breast Cancer Xenograft

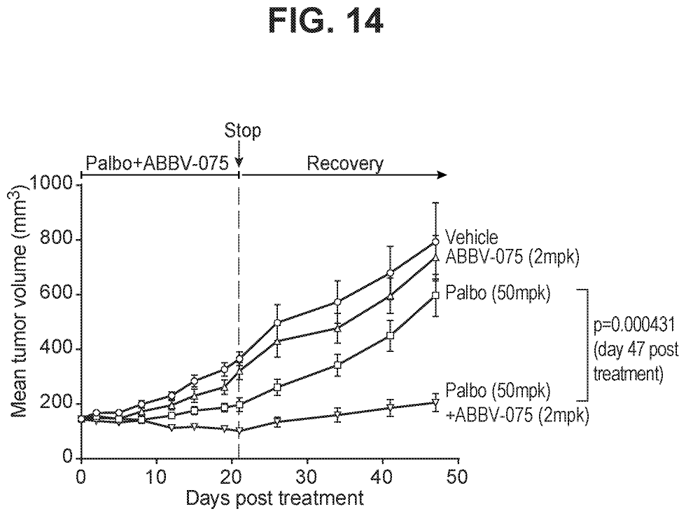

NOD scid gamma (NSG) mice (6-7 weeks old, Jax laboratory, Sacramento CA) were subcutaneously injected with 5×10⁶ MCF-7 cells (ATCC) with 50% matrigel. When MCF-7 xenografts reached a volume of -150 mm³, the tumor bearing mice were randomly assigned to four treatment groups, with each n=10 (vehicle, palbociclib (50 mpk), mivebresib (2 mpk), combination treatment with palbociclib (50 mpk) plus mivebresib (2 mpk)). Treatment was initiated after the randomization as indicated. Mice were treated once daily from days 0-20 and then tumors were allowed to recover until day 47. Mean tumor volumes (mm³) are averages of MCF-7 xenografts (n=10). Tumor growth inhibition curves are provided in FIG. 14.

All publications and patent applications cited in the specification are herein incorporated by reference in their entirety. Although the foregoing invention has been described in some detail by way of illustration and example, it will be readily apparent to those of ordinary skill in the art in light of the teachings of this invention that certain changes and modifications may be made thereto without departing from the spirit or scope of the appended claims.

1-20. (canceled)

21. A method of treating breast cancer or pancreatic cancer in a subject comprising administering to the subject an effective amount of palbociclib, or a pharmaceutically acceptable salt thereof, and an effective amount of a bromodomain and extra-terminal domain (BET) inhibitor.

22. The method of claim 21, wherein the BET inhibitor is an inhibitor of one or more of bromodomain-containing protein 2 (BRD2), bromodomain-containing protein 3 (BRD3), bromodomain-containing protein 4 (BRD4), or testis-specific bromodomain-containing protein (BRDT).

23. The method of claim 22, wherein the BET inhibitor is an inhibitor of BRD4.

24. The method of claim 22, wherein the BET inhibitor is an inhibitor of BRD2 and BRD4.

25. The method of claim 21, wherein the BET inhibitor is mivebresib, having the structure: 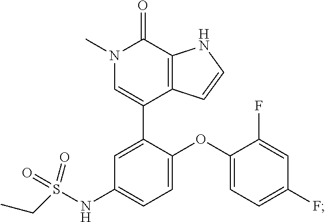 [SMILES: CCS(=O)(=O)Nc1ccc(Oc2ccc(F)cc2F)c(-c2cn(C)c(=O)c3[nH]ccc23)c1]
or
AZD5153 having the structure: 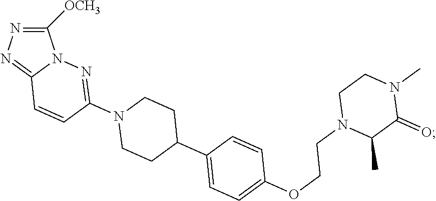 [SMILES: COc1nnc2ccc(N3CCC(c4ccc(OCCN5CCN(C)C(=O)[C@H]5C)cc4)CC3)nn12]
or a pharmaceutically acceptable salt thereof.

26. The method of claim 21, wherein the cancer is breast cancer.

27. The method of claim 26, wherein the breast cancer is hormone receptor positive (HR+), human epidermal growth factor receptor 2 negative (HER2−) breast cancer.

28. The method of claim 27, wherein the breast cancer is HR+, HER2− locally advanced or metastatic breast cancer.

29. The method of claim 27, wherein the method further comprises administering an endocrine agent.

30. The method of claim 29, wherein the endocrine agent is an aromatase inhibitor, a selective estrogen receptor downregulator (SERD), or a selective estrogen receptor modulator (SERM).

31. The method of claim 29, wherein the endocrine agent is letrozole or fulvestrant.

32. The method of claim 21, wherein the cancer is pancreatic cancer.

33. The method of claim 32, wherein the is pancreatic cancer is locally advanced or metastatic pancreatic cancer.
